(19) United States
(12) Patent Application Publication
(10) Pub. No.: US 2021/0321991 A1
(43) Pub. Date: Oct. 21, 2021

(54) Collection Swab

(71) Applicant: Applied Medical Resources Corporation, Rancho Santa Margarita, CA (US)

(72) Inventors: Patrick Elliott, Rancho Santa Margarita, CA (US); Thomas D. Wachli, Rancho Santa Margarita, CA (US); Ghassan Sakakine, Rancho Santa Margarita, CA (US); Dillon Davies, Rancho Santa Margarita, CA (US); Christopher Nguyen, Rancho Santa Margarita, CA (US); Chase Kingsbury, Rancho Santa Margarita, CA (US); Stephen Gyugyi, Rancho Santa Margarita, CA (US); Mark Leroux, Rancho Santa Margarita, CA (US); Matias Araya, Rancho Santa Margarita, CA (US); Thomas M.B. Roberts, Rancho Santa Margarita, CA (US)

(21) Appl. No.: 17/233,307

(22) Filed: Apr. 16, 2021

Related U.S. Application Data
(60) Provisional application No. 63/012,075, filed on Apr. 17, 2020.
(60) Provisional application No. 63/026,393, filed on May 18, 2020.

(51) Int. Cl. A61B 10/02

(52) U.S. Cl. CPC A61B 10/02

(57) ABSTRACT

The disclosed technology described pertain to collection swabs that are designed to capture and retain specimen, tissue, or fluids from a patient. The collection swabs are generally inserted into a desired location, such as a nasal cavity. During the process of insertion, a user maneuvers the collection swab and in the process dislodges and collects various specimen, tissue, or fluids from the patient. The collection swab utilizes specially designed distal or tip portions and/or sleeves or cages in order to capture and retain the specimen, tissue, and fluids. The various designs associated with the distal or tip portion and the sleeve or cages affect not only how much specimen, tissue, or fluids can be collected but also how much of the specimen, tissue, or fluids can be retained after the collection swab is removed from the patient. By obtaining the various specimen, tissue, or fluids, studies can be performed regarding the patient's health, for example, identifying the presence of specific virus such as COVID-19.

### CROSS-REFERENCE TO RELATED APPLICATIONS

The present application claims the priority benefit of U.S. provisional patent application 63/026,393 filed May 18, 2020 and U.S. provisional patent application 63/012,075 filed Apr. 17, 2020, the disclosures of which are incorporated herein by reference.

### BACKGROUND

The present application is directed to systems, methods and devices used in medical related testing and/or diagnostics and more particularly to systems, methods and devices to collect liquid and/or tissue samples to be used for, for example, testing or diagnosing patients or the like for a particular disease, illness, condition or predisposition.

### DESCRIPTION OF THE RELATED ART

Liquid and/or tissue specimens or samples may be extracted from a patient or the like through the mouth, nose, or other parts of the body. Collecting samples from the nose can be particularly challenging due to the varying depth necessary to reach an area to obtain a viable and accurate sample.

Once at the optimal area, sufficient liquid and/or tissue needs to be collected. Inadequate collection can cause inaccurate tests or diagnoses. Inadequate collection may also be a result of not collecting a sufficient amount of the desired sample within the allotted collection time.

Accelerating or increasing the collection of the sample thus may further ensure that the quantity and/or viability of the sample is optimally collected while also simultaneously reducing patient discomfort and operational difficulties or challenges. Retaining the sample to be contained or transferred and releasing a sufficient amount of the collected sample to be tested can also be a challenge.

### SUMMARY OF THE CLAIMED INVENTION

Disclosed herein are a variety of different collection swabs. The collection swabs are made of at least a handle portion that is used to maneuver the collection swab within a desired location of a patient and a distal or tip portion that is designed to collect specimen, tissue, or fluids located at the desired location of the patient such as a nasal cavity. The distal or tip portion is also designed to retain the specimen, tissue, or fluids collected from the patient after the collection swab is removed from the desired location.

Other embodiments of the collection swabs further include a sleeve or cage that is connected to and encompasses the distal or tip portion of the collection swab. The sleeve or cage provides further design space affecting how the collection swab collects and retains the specimen, tissue, or fluids being obtained from the patient. In some cases, the sleeve or cage can be designed to collect the specimen, tissue, or fluids and transfer the collected specimen, tissue, or fluids into an interior space of the sleeve or cage. This allows the enclosed distal or tip portion to capture and retain the specimen, tissue, or fluids from the desired space without having the distal or tip portion interact with the desired space directly. Alternatively, the sleeve or cage can also collect and retain any specimen, tissue, or fluid along with the distal or tip portion of the collection swab.

### BRIEF DESCRIPTION OF THE DRAWINGS

FIG. 1 is a side view of a shaft in accordance with various embodiments of the present invention.

FIG. 2 is a side view of a sleeve on a tip or distal portion of a shaft in accordance with various embodiments of the present invention.

FIG. 3 is a perspective view of a collection swab and a collection container in accordance with various embodiments of the present invention.

FIG. 4A-FIG. 40C provide various views of various embodiments of one or more of distal or tip portion and the sleeve or cage in accordance with various embodiments of the present invention.

### DETAILED DESCRIPTION

In accordance with various embodiments, a collection system is provided. In various embodiments, the collection system comprises a collection swab and a collection container. In various embodiments, the collection swab is a nasopharyngeal swab and the collection container is configured to receive a distal portion of the collection swab for the depositing of a collected sample, specimen, or the like. In various embodiments, the collection swab comprises a shaft and a cover or sleeve. In various embodiments, the collection swab comprises a shaft and no cover or sleeve. In various embodiments, the collection swab comprises a shaft with a distal end or portion with or without a sleeve. The sleeve is configured to retrieve a test sample from a patient's nasopharynx or other anatomical locations. Nasopharyngeal swabs are required to obtain patient nasal secretions to test for the presence of respiratory viruses such as COVID-19, and in accordance with various embodiments, the collection swab comprising the shaft and/or the sleeve provides such a nasopharyngeal swab.

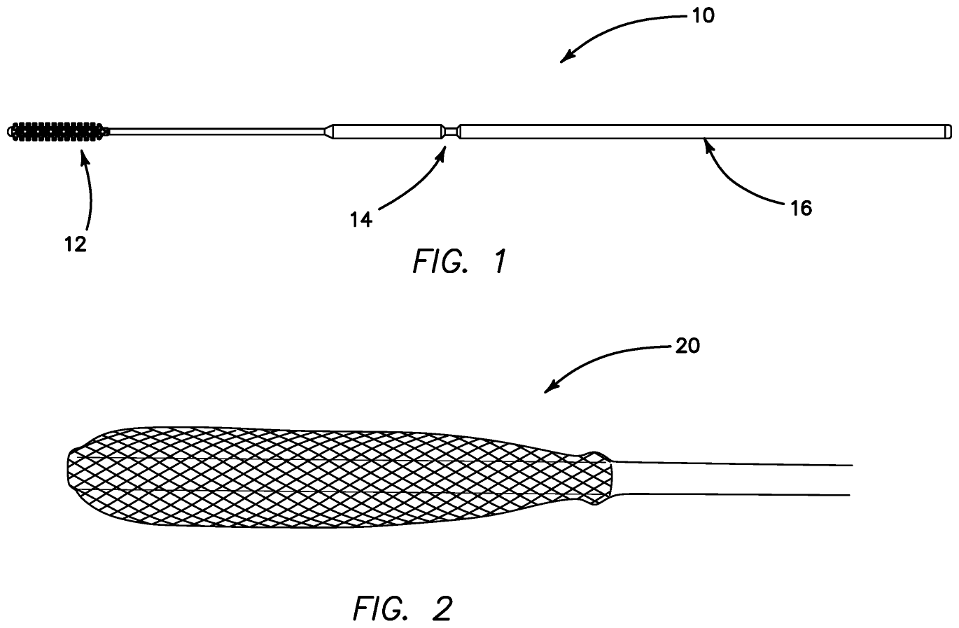

In accordance with various embodiments and as shown, for example in the figures of the present application, a collection system and/or various portions and embodiments thereof are provided. With respect to FIG. 1, an exemplary collection swab 10 is illustrated. A shaft of the collection swab 10 is provided having a distal or tip portion 12, a break portion 14, and a handle portion 16. In various embodiments, the shaft of the collection swab 10 is injection molded. Although the figure illustrates an embodiment of the shaft of the collection swab 10 which has the distal or tip portion 12, the break portion 14, and the handle portion 16, other embodiments are also possible where the shaft of the collection swab 10 comprises the distal or tip portion 12 and a proximal or handle portion 16 without the break portion 14.

With respect to the embodiment illustrated in FIG. 1, if the break portion 14 is provided in the shaft of the collection swab 10, such break portion is generally disposed between the distal or tip portion 12 and the handle portion 16. With respect to the distal or tip portion 12, in various embodiments, may have a shape different from the rest of the shaft of the collection swab 10. In various embodiments, the distal or tip portion 12 delimits a generally oval or oblate shape. The distal or tip portion 12 of the shaft can also comprise one or more protrusions or projections; one or more apertures, holes, divots and/or through-holes; or any combination thereof. In various embodiments, the distal or tip portion 12 comprises one or more gaps or spacing between the above-mentioned protrusions, apertures, through-holes, or any combination thereof.

In various embodiments, the distal or tip portion 12 is covered or otherwise includes a sleeve 20 (as illustrated in FIG. 2). The sleeve 20 can cover completely or partly encompassing the distal or tip portion 12. In various embodiments, the distal or tip portion 12 of the shaft comprises an intermediary portion connected to the handle portion 16. The distal or tip portion 12 may have a shape different from the intermediary portion and/or handle portion 16. In various embodiments, the intermediary portion and the handle portion 16 are sized and shaped differently from each other. In various embodiments, the intermediary portion has an outer diameter smaller than the handle portion 16 and/or the distal or tip portion 12. In various embodiments, the distal or tip portion 12 has or defines an outer diameter larger than the handle portion 16 and/or intermediary portion.

As noted above the distal or tip portion 12 can further comprises a sleeve or cover 20 as illustrated in FIG. 2. In various embodiments, the sleeve 20 comprises a plurality of filaments or braids, e.g., monofilament braids. In various embodiments, the plurality of filaments or braids are interwoven together to create a pattern such as a lattice work, a generally diamond shaped, or similar shaped openings. In various embodiments, the pattern creates or provides openings or passages between the interwoven filaments or braids. In various embodiments, the openings or passages provide access or passageways to the distal or tip portion 12 of the shaft.

In various embodiments, the sleeve 20 comprises one or more layers of one or more monofilament braids, one or more layers of a plurality of interwoven monofilament braids, and any combination thereof. In various embodiments, the sleeve 20 comprises a first layer of twenty-four interwoven monofilament braids covering or overlaying a second layer of twenty-four interwoven monofilament braids. In various embodiments, the layers and/or the plurality of braids of one or more of the layers are interwoven together to create a pattern such as a lattice work, a generally diamond shape, or similarly shaped openings. In various embodiments, the pattern creates or provides openings or passages between the layers of the interwoven braids. In various embodiments, the openings or passages between the layers of the interwoven braids provide access or passageways to the distal or tip portion 12 of the shaft.

In various embodiments, the sleeve 20 covers or is otherwise attached to the distal or tip portion 12 of the shaft. In various embodiments, the distal or tip portion 12 of the shaft comprises one or more protrusions and one or more apertures and/or through-holes. In various embodiments, the distal or tip portion 12 of the shaft has a diameter greater than a proximal end or proximal portion of the shaft. In various embodiments, the distal or tip portion 12 of the shaft has a shape that delimits or provides an outer profile or periphery of a generally oval or oblate shape. In various embodiments, the middle portion or near the middle portion of the distal or tip portion 12 of the shaft has or delimits a diameter greater than the outer diameter of the shaft.

In various embodiments, the sleeve 20 forms an enclosure encompassing the distal or tip portion 12 of the shaft. In various embodiments, the sleeve 20 comprises one or more layers of braided monofilaments. The sleeve 20 may have a tip that is formed using heat. In particular the heat may be used to form the tip of the sleeve 20 or at least a closed end of the sleeve 20. Tipped layers of the sleeve 20 are slipped over the distal or tip portion 12 of the shaft through an opposing or open end of the sleeve 20. The open end of the sleeve 20 is then attached to the shaft, for example using an adhesive such as UV-cured glue.

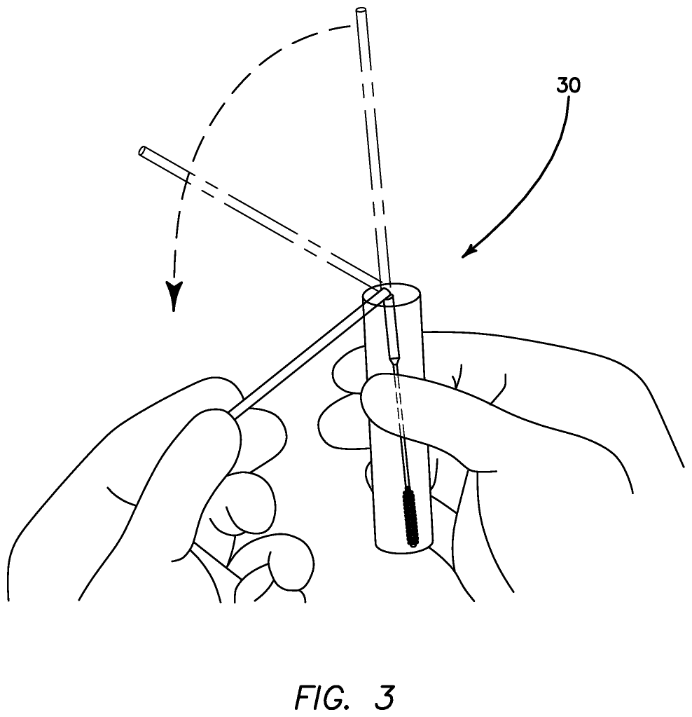

In various embodiments, as illustrated in FIG. 3, the shaft of the collection swab 10 comprises a break portion 14 separating the distal or tip portion 12 of the shaft from the proximal or handle portion 16 of the shaft of the collection swab 10. In various embodiments, the distal or tip portion 12 of the shaft is broken off at the break portion 14 (including portions up to the break portion). Afterwards, the separated portion can be placed into a container 30, vial or the like that provides a sterile viral transport medium to be stored and processed for diagnostic testing purposes.

In various embodiments, the break portion 14 is an attachment area or portion releasably coupling the distal or tip portion 12 to the handle portion 16 of the shaft. In various embodiments, a subsequent distal or tip portion 12 may be releasably attached to the handle portion 16 of the shaft.

In various embodiments, the collection swab length and diameter (i.e., geometry) allows for sufficient access to the desired specimen collection location. In various embodiments, the diameter of the portion between the handle portion 16 and the distal or tip portion 12 allows for bending to navigate the anatomy to access the desired collection location where the desired specimen may be located. In various embodiments, the break portion 14 allows for separation of the distal or tip portion 12 of the shaft associated with the collection swab 10 to be placed in a specimen collection container 30. In various embodiments, the break portion 14 facilitates a clean break of the distal or tip portion 12 of the shaft without additional fragments. In various embodiments, the break portion 14 and/or shaft of the collection swab 10 is made of polystyrene thereby facilitating in the clean break of the break portion 14.

In various embodiments, the distal or tip portion 12 has a geometry and associated sleeve 20 (e.g., braided layers) that facilitates in collecting samples or fluid collection by adsorption. Sample retrieval, in various embodiments, is also facilitated by the selection of hydrophobic materials of at least the distal or tip portion 12 and sleeve 20. For example, polystyrene and polyester are considered generally hydrophobic. Therefore, in accordance with various embodiments, the materials of the distal or tip portion 12 and sleeve 20 do not interact with the sample. Because the sample fluids are not being absorbed into the distal or tip portion 12 and/or sleeve 20 material, there is minimal or no concern of sample contamination at the time of sample collection.

In various embodiments, the spacing or gaps between the filaments (e.g., monofilaments) and/or the overlapping filaments delimiting the sleeve 20 and making up a sleeve surface and/or the voids in the geometry of the distal or tip portion 12 that causes the adsorption, capillary, and/or capture effect. The spacing or gaps between the filaments (e.g., the braided monofilaments lattice) of the sleeve 20 also allows for the fluid or sample to be adsorbed into the voids created in the distal or tip portion 12 geometry (e.g., protrusions, apertures, and the like). The underlying geometry of the distal or tip portion 12 is also configured such that the distal or tip portion 12 will not cause fluid or air to be trapped, such as in pockets, as the adsorption effect is advancing the fluid in the distal end through the openings or gaps between the monofilaments of the sleeve 20.

In various embodiments, the collection swab 10 provides an uptake volume of the fluid or specimen of about 20-100 uL. In various embodiments the collection swab 10 can also provide an uptake volume of the fluid or specimen of at least 100 uL and as high as 150 uL. In various embodiments, the uptake rate and/or volume can be varied by the sub structure of the distal or tip portion 12, for example by doing one or more of the following: varying the number, size, and shape of the voids in the sub structure and/or the sleeve 20, varying the spacing or gaps between the voids, the number, size and shape of each of the voids, varying the voids between the filaments and/or layers and/or the number, size and shape of overlapping layers associated with the sleeve 20. In various embodiments, the uptake rate and/or volume can be varied by the distance or space between the sleeve 20 and the sub structure of the distal or tip portion 12. In various embodiments, the uptake rate and/or volume can be varied by any combination of the above.

The combined underlying structure associated with the distal or tip portion 12 and braided layer(s) or sleeve(s) 20, in various embodiments, are also configured to facilitate sufficient release of the specimen or fluid for the same aforementioned reasons in regards to the hydrophobic nature of the material and the openings, spacing or gaps between or defined or delimited by the filaments of the layer(s) or sleeve(s) 20 as the distal or tip portion 12 geometry facilitates free flow of fluid having no pockets to trap fluid after it is placed in the sample container and vortexed for retrieval of the specimen for testing.

The combined underlying structure associated with the distal or tip portion 12 of the shaft and the braided covering or sleeve 20 in various embodiments is configured to not cause damage or irritation to the anatomy at the sample location. In accordance with various embodiments, the materials chosen allows for sterilization (e.g., with the use of e-beam, gamma, or the like). In various embodiments, the shaft of the collection swab 10 is made of polystyrene, the sleeve 20 is made of a polyester monofilament. Furthermore, a UV-cured glue can be used to connect the sleeve 20 and the shaft of the collection swab 10 together.

In accordance with various embodiment, the collection swab 10 is inserted into a patient's nasal cavity, ensuring that the area between the distal or tip portion 12 and the break portion 14 or intermediary portion is not touched during handling. The collection swab 10 is rotated to collect the sample, ensuring the distal or tip portion 12 only touches the suspected infections area to minimize potential contamination. After the specimen collection is completed, the collection swab 10 is placed into the collection container 30 and in various embodiments, a portion of the collection swab 10 is broken at the designated break portion 14. In accordance with various embodiments, the collection swab 10 may also be used as an applicator to collect a specimen in the same or similar fashion at other various anatomical locations.

In accordance with various embodiments, the collection systems described herein provide a sufficient or particular uptake volume of specimen (e.g., 20 uL-150 uL) or uptake of fluid in the distal or tip portion 12 of the shaft associated with the collection swab 10. In various embodiments, the collection swab 10 comprises of materials that does not interfere with diagnostic testing (e.g., Polymerase Chain Reaction Test) and/or may be sterilized. In accordance with various embodiments, the collection swab 10 provides efficient release of sufficient sample or fluid once the collection swab 10 is placed into the collection container 30. The collection container 30, in various embodiments, is vortexed with the collection swab 10 or portions thereof present to release the sample prior to the collection swab 10 removal from the collection tube 30. In accordance with various embodiments, the distal or tip portion 12 of the shaft associated with the collection swab 10 is atraumatic or does not cause irritation to the patient at the location where the specimen is taken.

In various embodiments, the sleeve 20 is position loosely around the distal or tip portion 12 of the shaft providing a gap to allow capture and subsequent release of the specimen. In accordance with various embodiments, the sleeve 20 and distal or tip portion 12 of the shaft is configured to provide an adsorption, capillary, and/or capture effect to collect the sample. In various embodiments, the pattern or gaps provided within the sleeve 20, distal or tip portion 12, gaps or spacing therebetween, or various combinations thereof provide an adsorption and capture effect to collect the tissue. In various embodiments, the pattern or gaps provided within the sleeve 20, distal or tip portion 12, gaps or spacing therebetween or various combinations thereof provide passageways to allow the complete or sufficient release or free passage of the specimen away from the distal or tip portion 12 of the shaft.

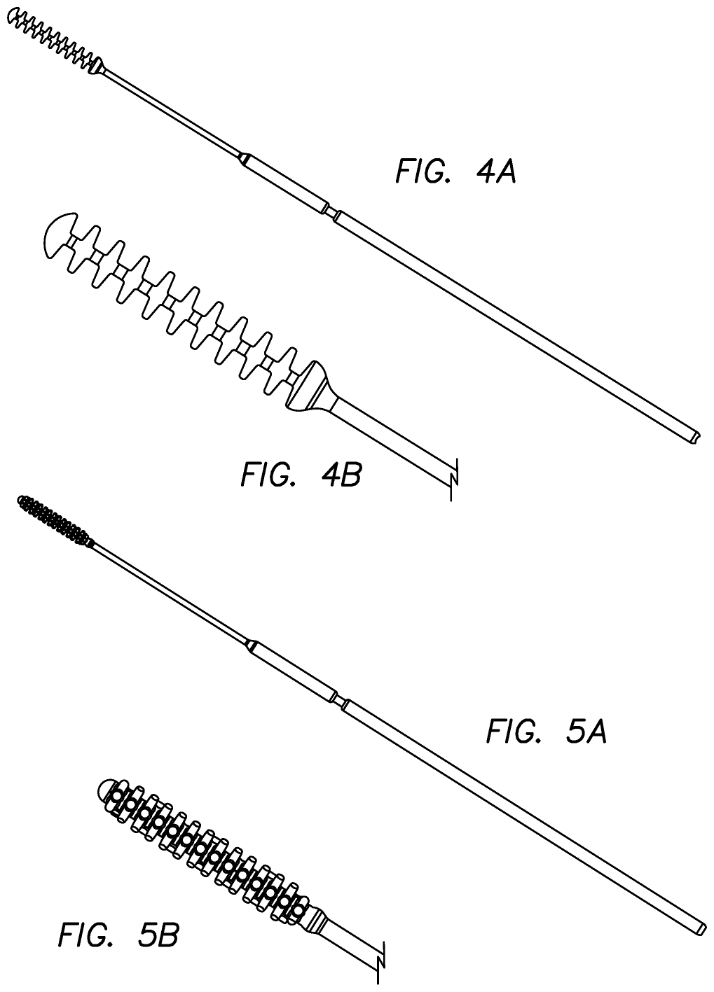

In accordance with various embodiments, the collection swab 10 comprises differing numbers of filaments (e.g., monofilaments), single or numerous filament layers, or any combinations thereof. In various embodiments, the sleeve 20 is attached by heat staking, adhesives, or the like, shrink tubing, or other mechanical attachments. In accordance with various embodiments, the collection swab 10 comprises a shaft with a single injection molded geometry without a sleeve. In accordance with various embodiments, the collection swab 10 comprises a shaft and one or more sleeves 20. In various embodiments, the collection swab 10 comprises one or more sleeves 20 being different or the same and/or be made of an absorbent material. In various embodiments, the distal or tip portion 12 is configured to have tiered type geometry as shown for example in FIGS. 4A-4B and one or more layers of an interwoven monofilament braid covers or encompasses the distal or tip portion 20 or post.

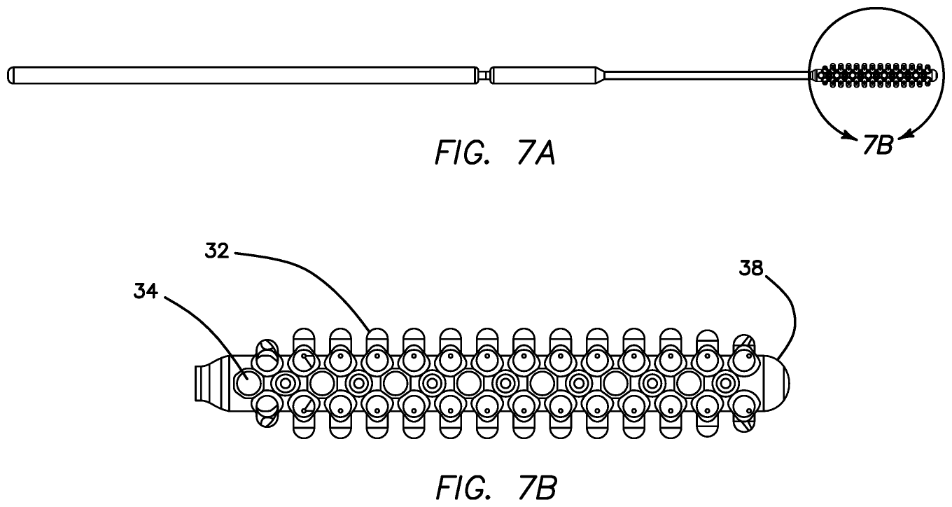

With reference to each of the figures in this application, a cover or sleeve (hereinafter referred to as a cage) are illustrated in accordance with various embodiments of the invention. The cage 20 is adhered to, mechanically snapped onto, or otherwise attached to the shaft of the collection swab. In various embodiments, the cage and shaft of the collection swab are integrated forming a monolithic structure. The shaft in various embodiments includes a distal or tip portion, inner geometry, extension, or post with a specified geometry including one or more projections, protrusions, ribs, apertures, divots, thru-holes (herein generally referred to as “apertures”) and/or various combinations thereof. In various embodiments, the projections, protrusions and/or ribs are curved or generally arcuate to maximize surface area for specimen/tissue collection and/or avoid traumatic contact with the collection site and/or the cage. As seen in the FIG. 4A-40C, various embodiments are illustrated. For example, some distal or tip portions may have apertures 34 that are spaced along the length of the post interspersed with the protrusions 32 (e.g., as shown in FIG. 7B, 10D or 12D).

In various embodiments, the protrusions 32 associated with the distal or tip portion are generally uniform in height and in spacing therebetween (see FIG. 7B). In various embodiments, the protrusion 32 extends perpendicularly from the extension. In various embodiment, one or more protrusions 38 are aligned with the extension and abuts or is spaced from the distal most end of the cage. In various embodiments, the aligned protrusion 38 is enlarged covering or capping the end of the extension and/or the end of the cage. In various embodiments, the proximal most protrusion and/or the distal most protrusions are shorter than the protrusions therebetween, e.g., as shown in FIG. 7B, 10D or 12D, providing a proximal taper and/or a distal taper to facilitate collection.

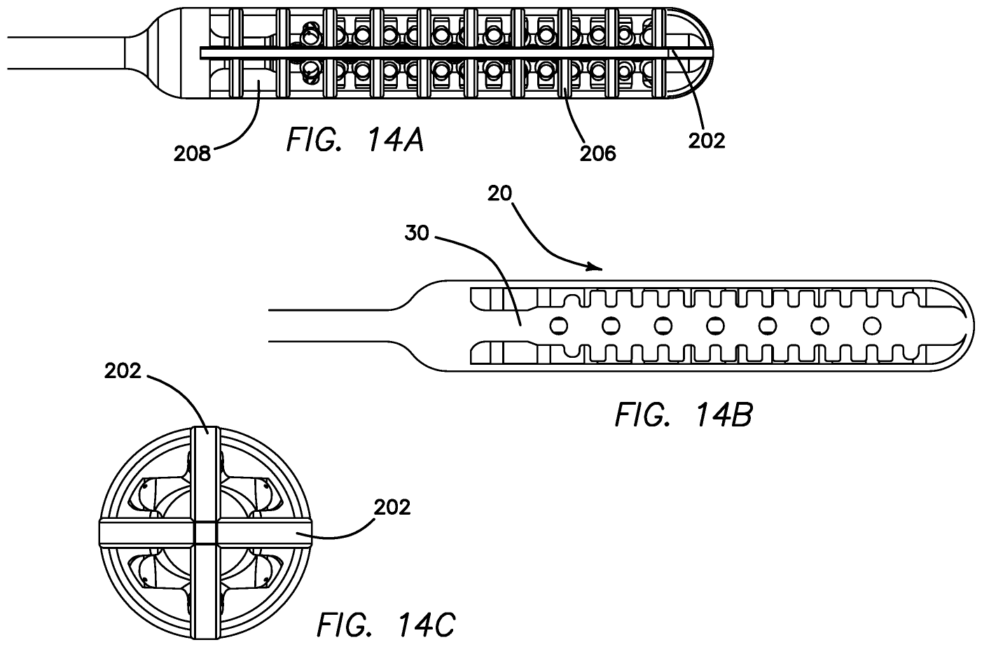

In various embodiments, the cage includes one or more panels, ribs, and/or braids. These features are capable of providing a specific operational or clinical outcome such as a desired capture or adsorption rate, retention, and/or release, and/or ease operation or manufacturing processes. Similarly, in various embodiments, the cage includes one or more spaces or openings and provides a specific operational or clinical outcome such as a desired capture or adsorption rate, retention and/or release, and/or ease operation or manufacturing processes. In various embodiments, as shown for example, in FIG. 14A, the ribs 206 are parallel to each other and uniformly spaced from each other. In various embodiments, the cage comprises an intertwined braid 24 and/or one or more layers of braids 24, 26. In various embodiments, the cage may comprise a combination of panels, ribs and braids, and/or various layers thereof. In various embodiments, the panels, ribs, or braids are curved, partially or fully, extending circumferentially around the cage. The panels, ribs, or braids in various embodiments may be uniform in thickness and/or width. In various embodiments, one or more panels or ribs 206 extend perpendicularly relative to a longitudinal axis of the shaft spaced longitudinally or lengthwise along the cage and/or one or more ribs 206 extends parallel, longitudinally or lengthwise along the cage. In various embodiments, the spacing 208 between the ribs and/or panels can vary. In various embodiments, the spacing 208 can range from about 0.05 inches to 0.110 inches.

In various embodiments, outer surface or surfaces of the panel, rib, braid, or combinations thereof are arranged to contact the desired patient's area and in particular contact the desired specimen or tissue such that the specimen/tissue collects and travels along the outer surface and/or ingress through the spaces or openings defined by a pattern or layout of the arranged panels, ribs, and/or braids, and/or between consecutive panels, ribs, and/or braids. As such, in various embodiments, the one or more spacings or openings between the one or more panels, ribs, braids, or any combination thereof, provides access of the ingress of the tissue or specimen within the confines or coverings delimited by the cage and is temporarily entrapped or captured within the confinement, the extension, or both. Likewise, the spacing or opening between the ribs, braid, or braids provide eventual egress of the tissue or specimen upon retrieval by vortex mechanisms, or the like for diagnostic or testing purposes. As such, in accordance with various embodiments, as shown for example throughout the figures, the one or more panels, ribs, braids, or combinations thereof and/or spaces or openings defined thereby can vary in number, size, alignment and/or shape and/or disposed along all, some or in clumps, with spacing therebetween uniform and/or varied, as desired to provide a specific operational or clinical outcome, e.g., desired capture or adsorption rate, temporary retention and/or release and/or ease operation or manufacturing processes.

In accordance with various embodiments, as shown for example throughout the figures, the one or more protrusions, projections, or ribs can vary in number, size, alignment and/or shape and/or disposed along all, some or in clumps, with spacing therebetween uniform and/or varied, as desired to provide a specific operational or clinical outcome, e.g., desired capture or adsorption rate, retention and/or release, reducing or eliminating air traps and/or supporting the cage, and/or ease operation or manufacturing processes. Likewise, the projections, protrusions or ribs are spaced or abut portions of the cage and/or are disposed within the spaces or openings of the cage. Similarly, the apertures, divots and/or thru-holes can vary in number, alignment, size and/or shape as desired to provide a specific operational or clinical outcome, e.g., desired capture or adsorption rate, retention and/or release and/or reducing or eliminating air traps, and/or ease operation or manufacturing processes.

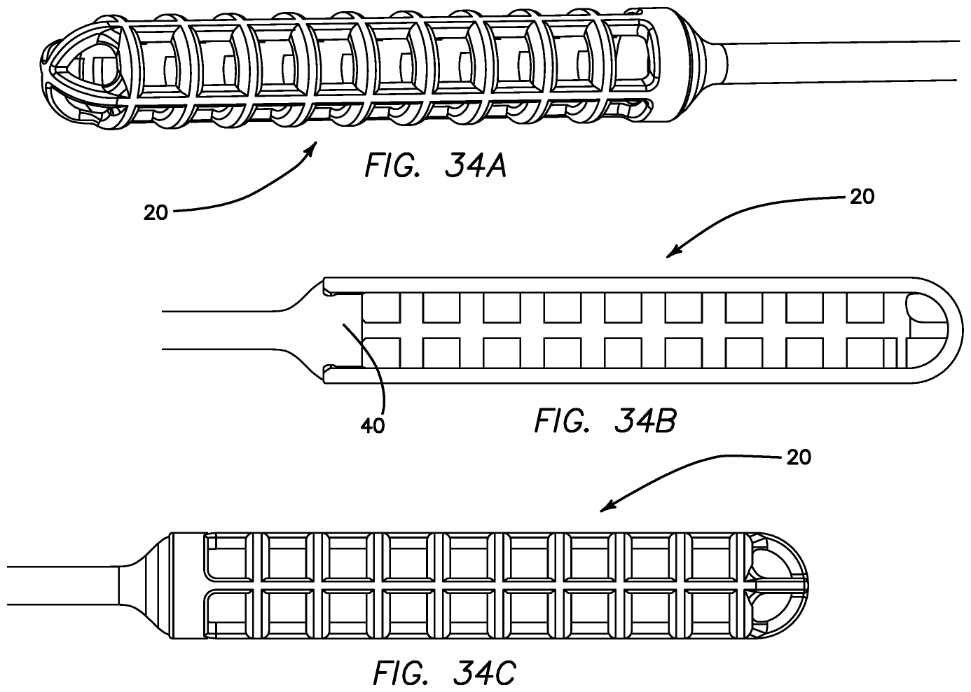

In various embodiments, the post or extension 30 may extend from a support or bolster 40. The bolster 40 is an enlarged portion at the distal end of the shaft and provides a support or attachment useful or assisting in the connection of the cover, sleeve or cage to the shaft. The bolster 40 in various embodiments includes one or more channels or cutouts 42 disposed around the periphery of the bolster to provide a snap connection or mating connection with one or more projections or tabs extending from a proximal end of the cage and similarly disposed around the periphery of the cage. In various embodiments, the cage provides the one or more cutouts or channels 211 and the bolster provides the one or more tabs or projections 44 or either provides combinations thereof. In various embodiments, protrusions 301 engage, snap or otherwise connect the cage within spaces between the ribs 206. In various embodiments, the cage comprises a proximal support ring having the one or more cutouts, channels, tabs, projections, or any combination thereof to mate with their like counterpart of the bolster. In various embodiments, the support ring includes one or more slots or apertures to provide a path or inlet for adhesive to be added to enhance the connection of the cage to the shaft. In various embodiments, the bolster provides a generally flat surface to provide a stable or uniform connection for the attachment of the cage. In various embodiments, the flat surface, channel, or protrusions include adhesive or the like to connect or bond the cage to the bolster 40. In accordance with various embodiments, an extension does not extend from the bolster 40 and as such the cage 20 encompasses or delimits an empty space, as shown for example in FIGS. 34A-36E.

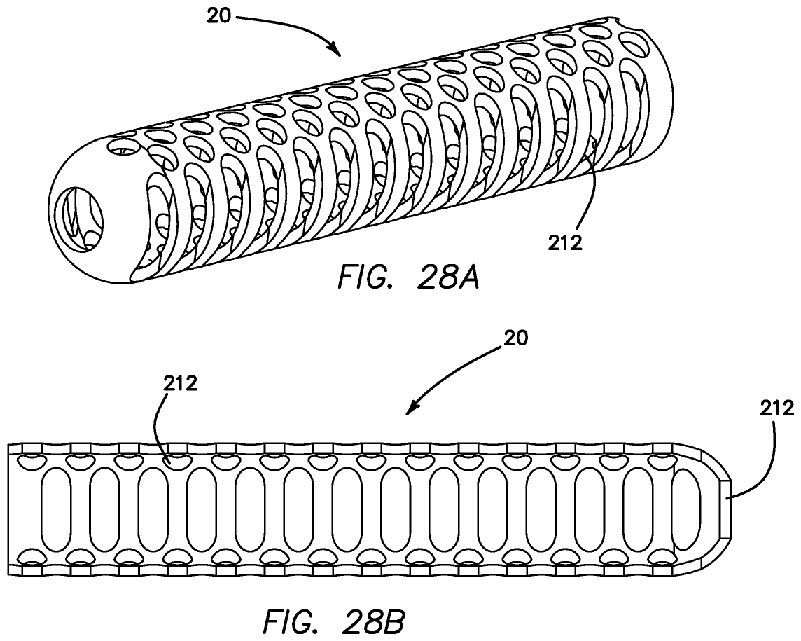

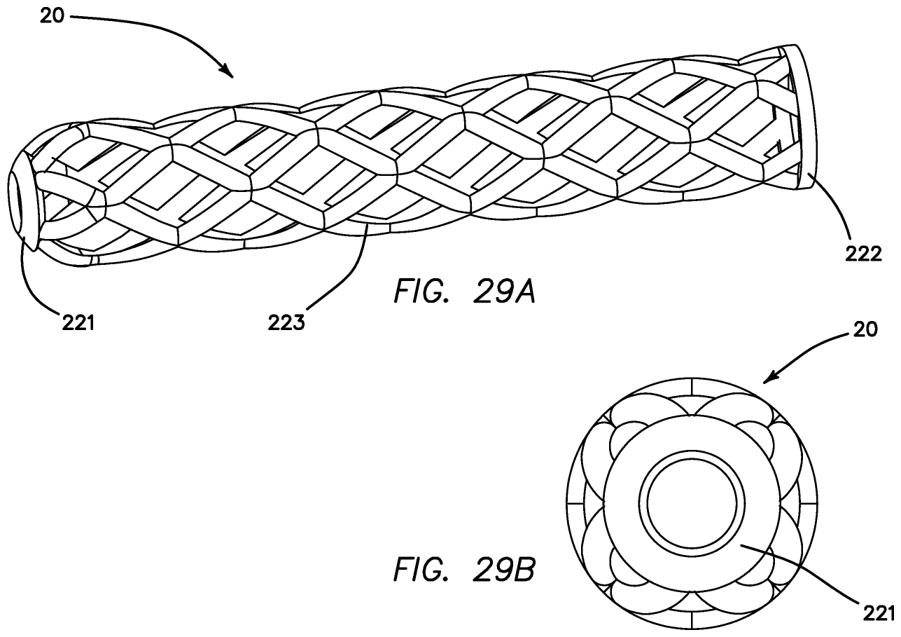

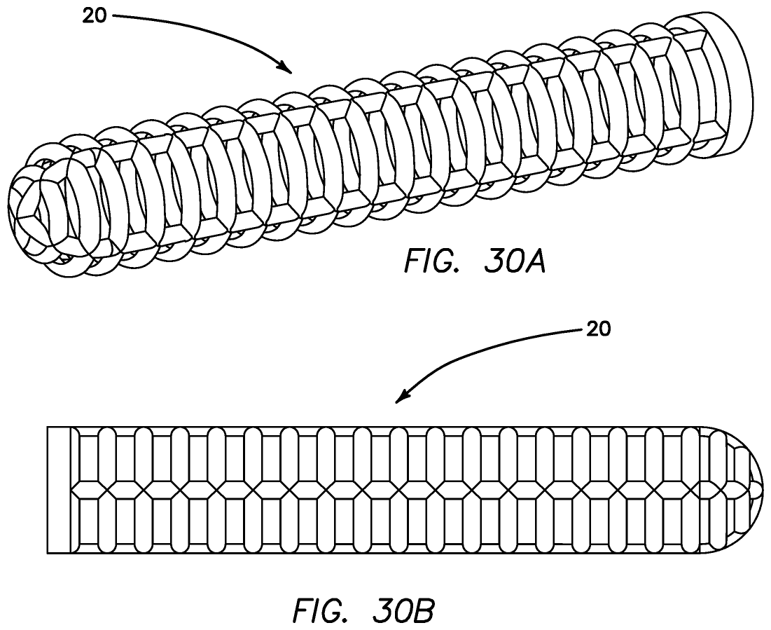

In various embodiments, the cage generally defines a cylindrical shape with an enclosed rounded or blunt tip. In various embodiments, the cage is flexible or elastic deformable to contact one or more of the protrusions of the extension, reducing potential discomfort or trauma at the collection site. In various embodiments, the cage is a cylindrical cover having one or more apertures, openings, or slots 212 therethrough as shown for example in FIGS. 28A-B. In various embodiments, as shown for example in FIGS. 29A-B, the cage comprises a distal ring and a proximal ring 222 with intertwined or laced ribs 223.

In the figures present in the application, various views of various embodiments of the cover, sleeve, cage 20 and/or distal portion of the shaft, extension or post along with various combinations thereof are shown. It should be noted that although various combinations are shown in these figures and described throughout the application, different embodiments of the sleeve/cage 20 and/or extension/post can be interchanged, combined and/or replaced to provide a desired operational outcome, e.g., desired capture or adsorption rate, retention and/or release, and/or ease operation or manufacturing processes. In accordance with various embodiments described throughout and various combinations thereof, collection swabs are described in which the distal portion of the shaft, extension or post provides inner swab geometry or structures and/or the cover, sleeve, cage provides an outer swab geometry or structures.

In various embodiments, the combination of the inner and outer geometry/structure, including the lack of one or the other, a balance is maintained or provided between specimen/tissue/fluid adsorption or capture volume and/or rate, along with air displacement, e.g., avoiding or removing air traps, and temporary specimen/tissue/fluid retention. In accordance with various embodiments and combinations thereof, the collection swab and in particular, the distal portion of the shaft, extension or post and/or the cover, sleeve and/or cage are made of material that has adsorptive properties but no or negligible absorptive properties (e.g., unlike a cotton swab) and as such facilitates ultimate release and/or retrieval of specimen/tissue/fluid for diagnostics and/or testing. In accordance with various embodiments and combinations thereof, proximal or handle portions of the collection swab as described throughout and/or unfeatured shafts, may be attached, structurally, mechanically, adhered or otherwise connected to the various embodiments and combinations thereof of the distal portion of the shaft, extension or post and/or the cover, sleeve and/or cage 20.

In the following sections, further details will be provided for each of the various embodiments alluded to above. In particular, the various embodiments, as discussed in FIG. 4A-40C, provide different features/limitations associated with one or more of the distal or tip portion of the shaft and/or the cover or cage. In some embodiments of the collection swab, the shaft of the collection swab may have one or both of the distal or tip portion and the cover/cage. The various embodiments, with the variations provide therein, each may provide different influences on how the collection swab absorbs or captures a specimen/tissue/fluid, affect a volume and/or rate by which the specimen/tissue/fluid can be collected, and provide different ways to temporarily retain the specimen/tissue/fluid (and assist in the retrieval of the specimen/tissue/fluid from the collection swab). Other affects to the collection swab can also be realized based on the variations discussed below illustrated in the figures within the present application.

With respect to FIG. 4A and FIG. 4B, a variation of the collection swab is illustrated. Specifically, FIG. 4A illustrates an exemplary collection swab while FIG. 4B illustrates a closer image of the distal or tip portion of the collection swab. Although FIG. 4A illustrates a collection swab with a break portion, there may be similar embodiments where the collection swab does not have the break point as illustrated.

With reference to FIG. 4A and FIG. 4B, the collection swab does not have a cover or cage encasing the distal or tip portion of the shaft associated with the collection swab. Furthermore, the shape of the distal or tip portion has a number of protrusions perpendicular from the shaft of the collection swab. In between each of the protrusions are a number of grooves. Although the figure shows these protrusions in two-dimensions, in reality, the protrusions are circular in three-dimensions. The end result of the combination of protrusions and grooves provide a “honey-dipper” like shape.

With the particular shape, as illustrated in the figure, the protrusions of the collection swab can be used to scrape/collect the specimen/tissue/fluid from the desired area (e.g., nasal cavity) of the patient as the collection swab is inserted into the desired area and/or moved around in the desired area. The specimen/tissue/fluid that are obtained from the protrusions can then be deposited within the grooves between each of the protrusions of the collection swab. When the collection swab is removed, the specimen/tissue/fluid is removed from the desired area.

As discussed above, the specimen/tissue/fluid collected via the collection swab can then be stored (along with part or the entirety of the collections swab) within a collection container. As discussed above, the collection container provides a secure/safe means of carrying the specimen/tissue/fluid as it is being transported or at least until the specimen/tissue/fluid can be used (e.g., testing).

Next, FIG. 5A and FIG. 5B illustrates another variation with the collection swab. The collection swab may be similar to the collection swab in FIG. 4A and FIG. 4B except for the features associated with the distal or tip portion used to collect the specimen/tissue/fluid. Whereas FIG. 4A and FIG. 4B utilized alternating protrusions and grooves, the collection swab of FIG. 5A and 5B uses protrusions with through holes. A further detailed illustration of this embodiment can also be seen in FIG. 7A and FIG. 7B.

The distal or tip portion may have a number of protrusions 32 with some or all of the protrusions having a through-hole located at the outer-most tip of the protrusion. There may also be additional apertures 34 located at the distal or tip portion positioned between the protrusions 32. In addition protrusions may also be located at the distal most point of the shaft 38.

Each of the protrusions and/or apertures 32, 34, 38 are configured to facilitate in the collection of specimens/tissues/fluids when the collection swab is inserted into a desired location. A benefit for the through-holes associated with the protrusions 32 may be to allow for the breaking of surface tensions as the collection swab is inserted and maneuvered within the desired location (e.g., nasal cavity). Furthermore, the through-holes may also provide benefits during the collection (e.g., absorption) and retaining of the specimen/tissue/fluid as the specimen/tissue/fluid may be captured within the protrusions 32. The specimen/tissue/fluid can be stored within the protrusions 32 until removed at a later time, for example, via the use of centrifugal force or by shaking the collection swab to dislodge the specimen/tissue/fluid from the protrusions.

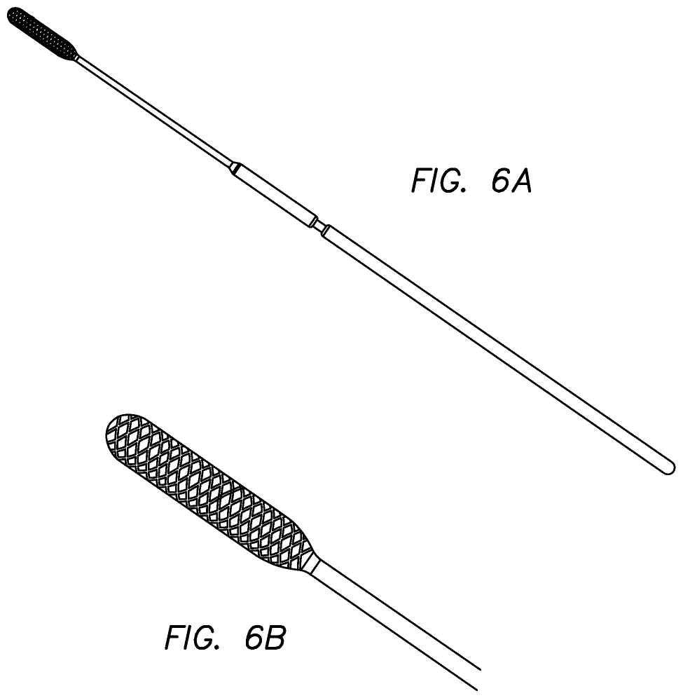

FIG. 6A and FIG. 6B illustrates an embodiment of a sleeve or cage. The sleeve or cage (as illustrated in the figure) may be used in connection with any distal or tip portion described within this application such as those that were illustrated in FIG. 4A/FIG. 4B and FIG. 5A/FIG. 5B. Furthermore, the sleeve or cage, as illustrated in FIG. 6A and FIG. 6B may be used in connection with any distal or tip portion described below. When used in connection with a distal or tip portion, the sleeve or cage covers/surrounds the entirety of the distal or tip portion.

As discussed above, the sleeve or cage can be used to facilitate in the collection of specimen/tissue/fluid from the desired location (e.g., nasal passage). In FIG. 6A and FIG. 6B, the sleeve or cage are made of interweaving elements which form diamond-shaped openings. These diamond-shaped openings allow access from the exterior of the sleeve or cage to an interior of the sleeve or cage where, for example, the distal or tip portion of the collection swab is located. It should be noted that different arrangements may be possible for the sleeve or cage (as discussed below in the following figures). Furthermore, there may be embodiments where the collection swab may comprise a sleeve or cage without a distal or tip portion located within.

As the collection swab with the sleeve or cage is inserted and maneuvered in the desired location, it may be possible to dislodge and subsequently collect specimen/tissue/fluid as the surface of the sleeve or cage come into contact with the desired location. The sleeve or cage may be capable of retaining some of the specimen/tissue/fluid after collecting from the desired location. The sleeve or cage may have features or be made of materials that allow for the specimen/tissue/fluid to be retained by the sleeve or cage. In some cases, the specimen/tissue/fluid may also be pulled into the interior of the sleeve or cage thereby coming into contact with the distal or tip portion of the shaft associated with the collection swab (if present). The specimen/tissue/fluid may then attach itself to the distal or tip portion and be retained within the sleeve or cage in this manner as well.

The sleeve/cage can be used as a way to retain and transport the specimen/tissue/fluid collected from a patient. When the specimen/tissue/fluid is ready to be retrieved for use (e.g., testing), a user may use centrifugal force or by shaking the collection swab to dislodge the specimen/tissue/fluid from the sleeve or cage.

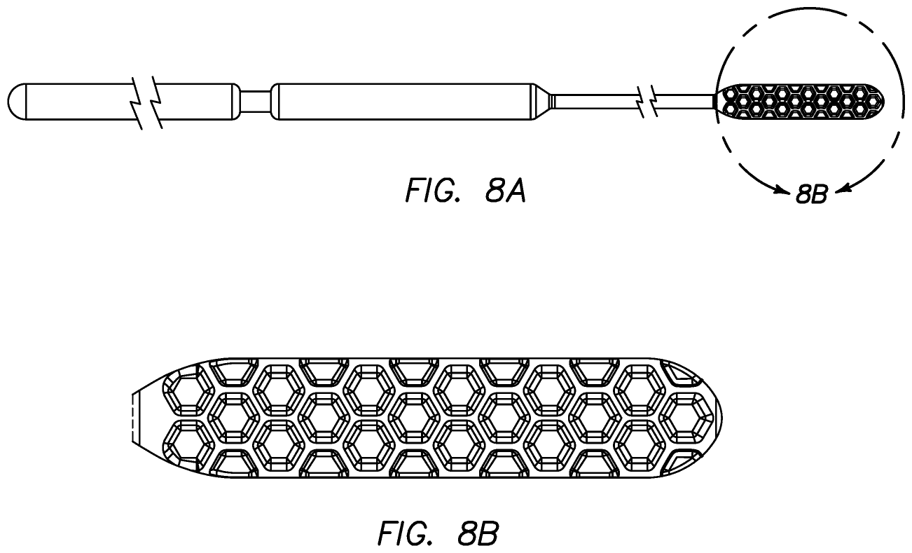

Turning now to FIG. 8A and FIG. 8B, another variation for the sleeve or cage is illustrated. The sleeve or cage can be manufactured, for example via injection molding, to have a surface having a number of access or passageways of a specific shape. With this figure, the access or passageways for the sleeve or cage can have a hexagonal shape. Furthermore, the access or passageways in this embodiment (as well as other embodiments discussed int his application) may be evenly spaces throughout the entirety of the surface of the sleeve or cage. In other embodiments, however, different arrangements of the access or passageways may be possible such as having some closer in proximity to each other in one portion of the sleeve or cage and having other access or passageways farther away from each other in a different portion of the sleeve or cage.

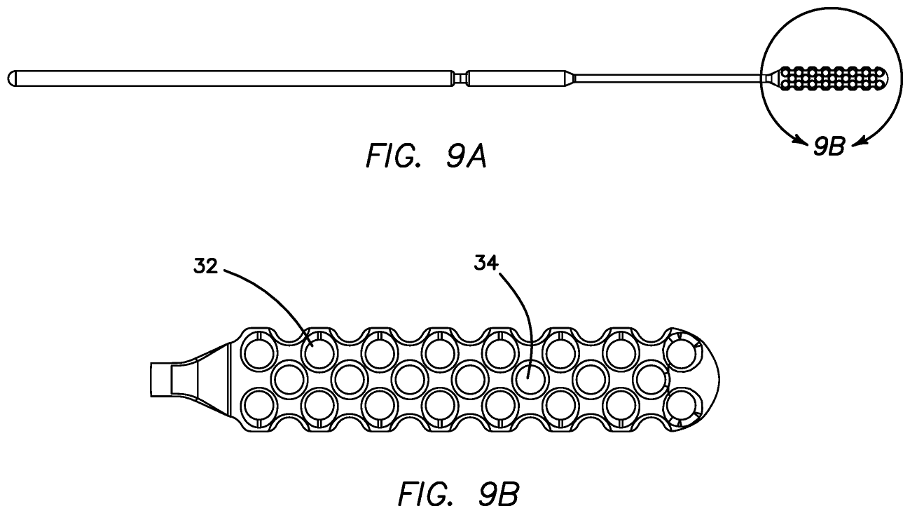

With respect to FIG. 9A and FIG. 9B, another variation of the distal or tip portion is illustrated. In particular, the distal or tip portion illustrated in the figure has a number of protrusions 32 that are evenly spaced throughout the sleeve or cage. In addition to the protrusions 32, some or all of the protrusions 32 may further have apertures 34 which can be separately attached onto the protrusions 32. In some cases, the protrusions 32 and the apertures 34 may be manufactured (e.g., molded) as a single component. In either case, the apertures 34 added to the protrusions 32 can assist in collecting the specimen/tissue/fluid as the collection swab is inserted/maneuvered in the desired location.

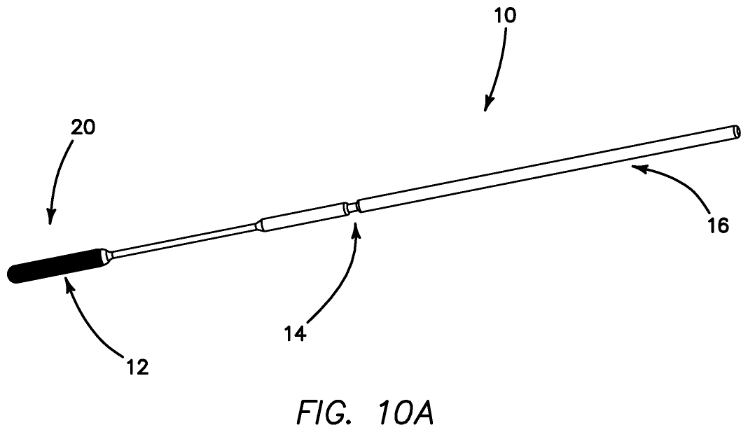

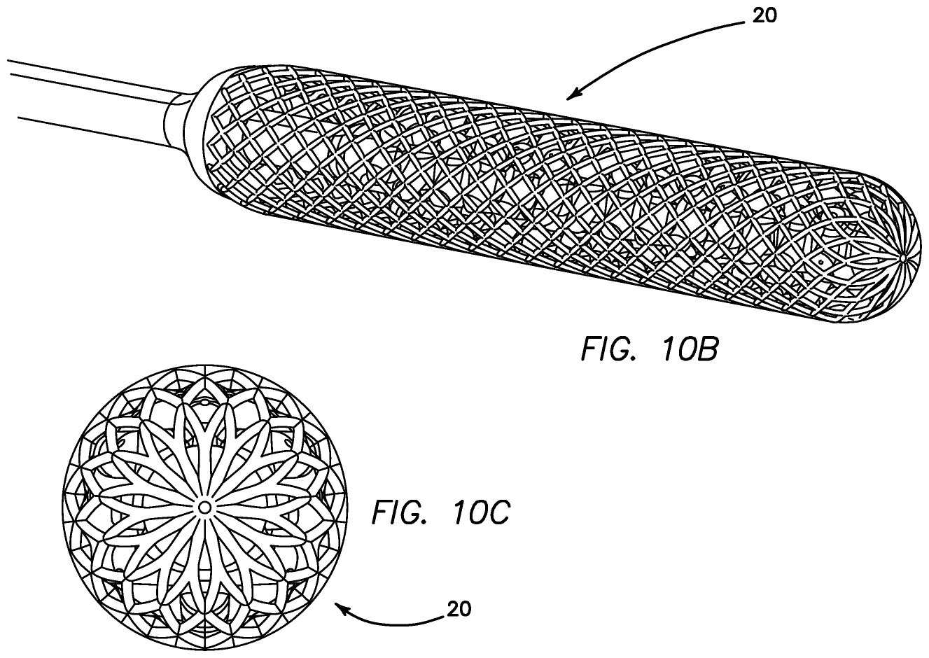

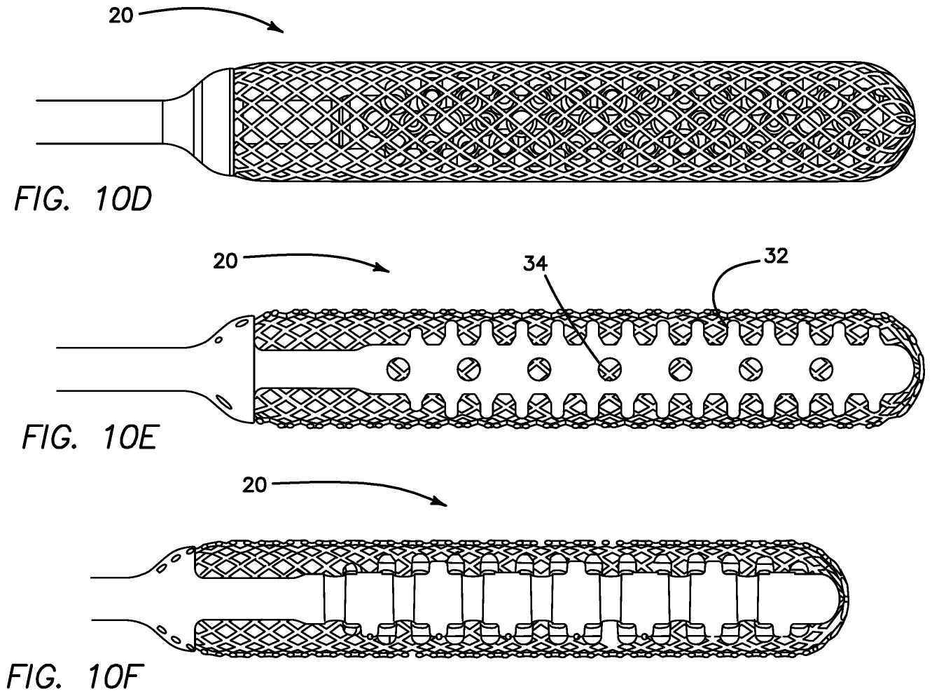

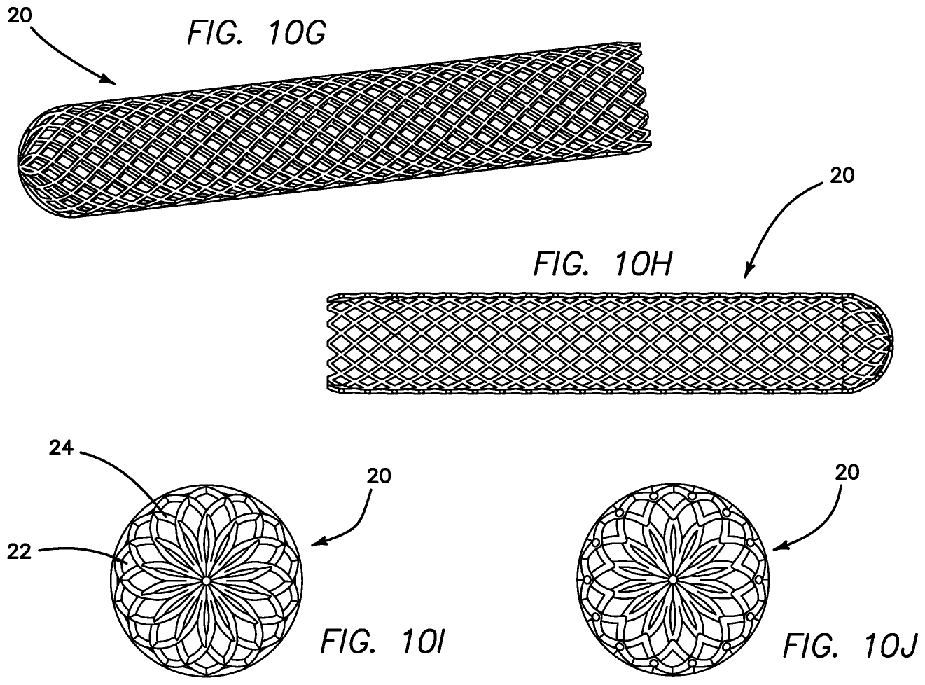

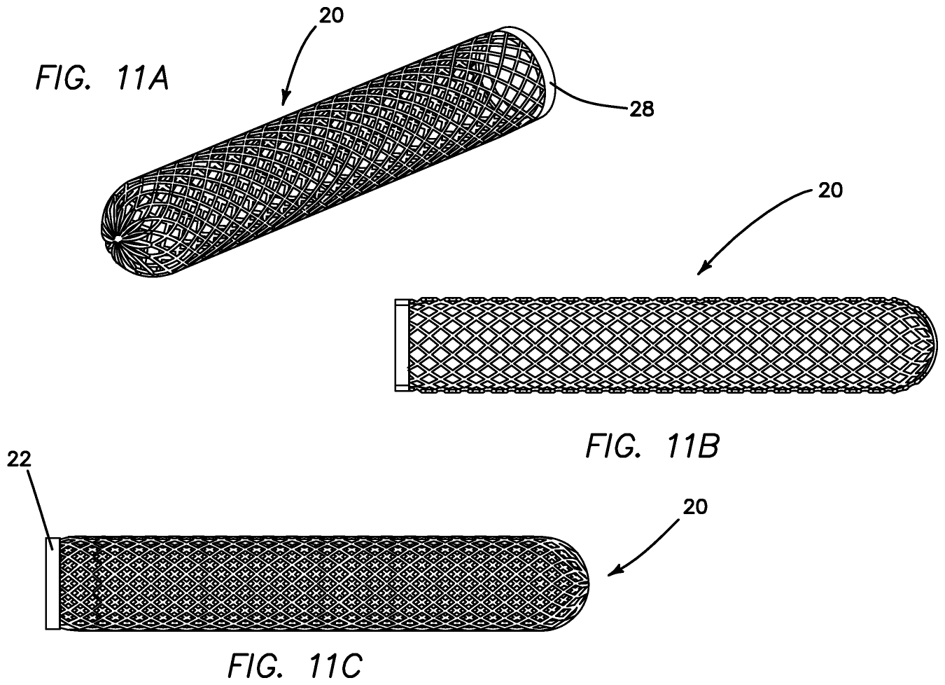

With respect to FIG. 10A-FIG. 10J, the figures illustrate additional different sleeves or cages that can be used in connection with the collection swab. FIG. 10A illustrates an overall design of the collection swab 10, similar to other embodiment described above. The collection swab 10 has a sleeve or cage 20 at the distal end of the collection swab 10 covering the distal or tip portion 12 of the shaft associated with the collection swab 10. The collection swab 10 also has a handle portion 16 and break portion 14 between the distal or tip portion and the handle portion 16. As described above the handle portion 16 facilitates in the maneuvering/positioning of the collection swab 10 in the desired location. A user would hold the handle portion 16 and maneuver the collection swab in order to collect the specimen/tissue/fluid. The break portion 14 would facilitate in the subsequent storage once the specimen/tissue/fluid has been collected, respectively by allowing the user to break off the portion of the collection swab (e.g., distal or tip portion) and store that portion in the collection container.

FIG. 10B and FIG. 10C illustrate an exemplary design of the sleeve or cage whereby the materials can be interwoven (or molded) in order to form diamond-shaped access or passageways. Specifically with FIG. 10C, the design at the distal most point of the sleeve or cage can be different than the design at other portions of the sleeve or cage. In particular, the materials that are interwoven (or at least molded) can all come to a point at the distal most point of the sleeve or cage.

FIG. 10D-FIG. 10F illustrate an exemplary embodiment combining both the sleeve or cage with a distal or tip portion that would be enclosed within. FIG. 10D provides an outside view of the sleeve or cage which covers the entirety of the distal or tip portion. FIG. 10E and FIG. 10F illustrate a cross-section of the sleeve or cage whereby the enclosed distal or tip portion of the shaft associated with the collection swab can be seen. The cross-section illustrates how the distal or tip portion (even with its various protrusions and apertures) are completely enclosed within the sleeve or cage such that no portion of the distal or tip portion can be seen extending past the sleeve or cage. However, there may be embodiments where this is not the case and such embodiments are possible.

FIG. 10G-FIG. 10J illustrate additional alternative designs for the sleeve or cage different from those provided earlier in FIG. 10B and FIG. 10C. Although the majority of the body of the sleeve or cage may have a similar design with respect to how the access or passageways are arranged, the distal most point of the sleeve or cage can be provided a different design having different access or passageways. Depending on the type of specimen/tissue/fluid being collected and how such specimen/tissue/fluid are collected, having the access or passageways positioned at the distal most point of the sleeve or cage can provide a means of collecting the specimen/tissue/fluid as the collection swab is inserted into the desired location or pressed against surfaces (e.g., tissue walls).

FIG. 11A-FIG. 11C illustrates various perspectives of a sleeve or cage having a proximal support ring 28. The proximal support ring 28, as illustrated in the figures, is attached to the proximal end of the sleeve or cage. The proximal support ring 28 provides a surface that can be used to connect the sleeve or cage to the shaft. The shaft may have a corresponding component (e.g., bolster) that can provide a surface for the proximal support ring 28 to attach to. The connection between the proximal support ring 28 and the bolster may involve the use of a variety of different elements to provide for a secure attachment. Exemplary elements may include adhesives, slots, apertures, or the like which can be used to connect and lock the proximal support ring 28 and the bolster together. In some variations, the cage my be removable from the collection swab.

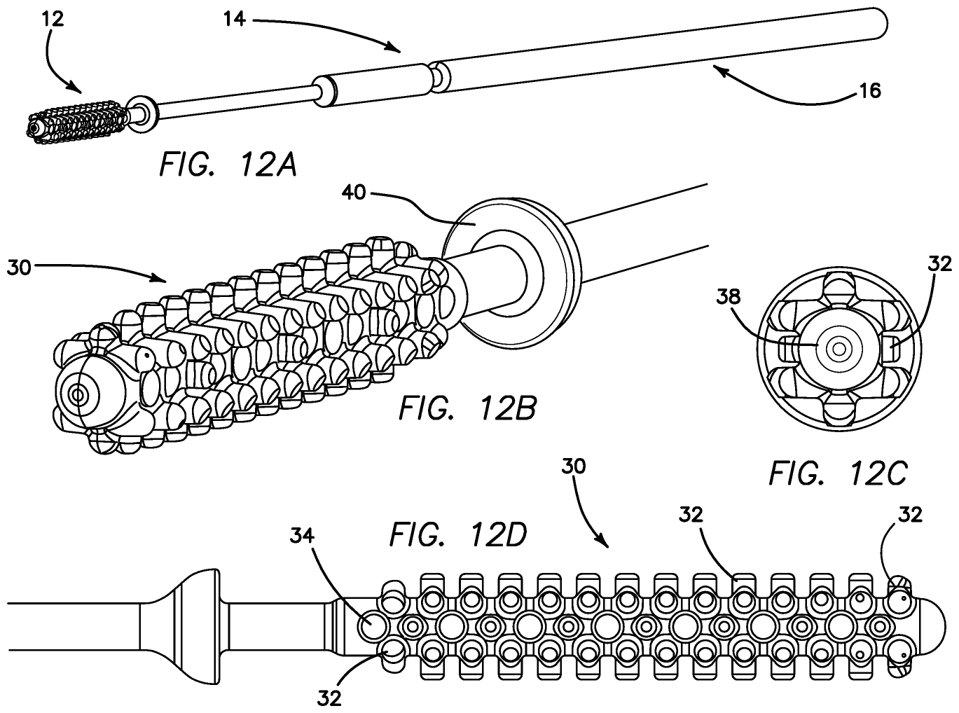

FIG. 12A-FIG. 12D illustrate different views of an exemplary collection swab with a distal or tip portion having an arrangement of protrusions and apertures. In particular, the exemplary collection swab illustrated in the figure can be seen as a combination of features described above with respect to FIG. 7A/FIG. 7B and FIG. 9A/FIG. 9B. The distal or tip portion, as seen in FIG. 12A-FIG. 12D has a combination of protrusions with through holes as well as protrusions with apertures. Furthermore, the height of the protrusions are not uniform. Some protrusions (with the associated apertures) extend higher from the shaft compared to the protrusions with the through holes. In addition, the distal or tip portion has a protrusion at the distal most point of the distal or tip portion 38.

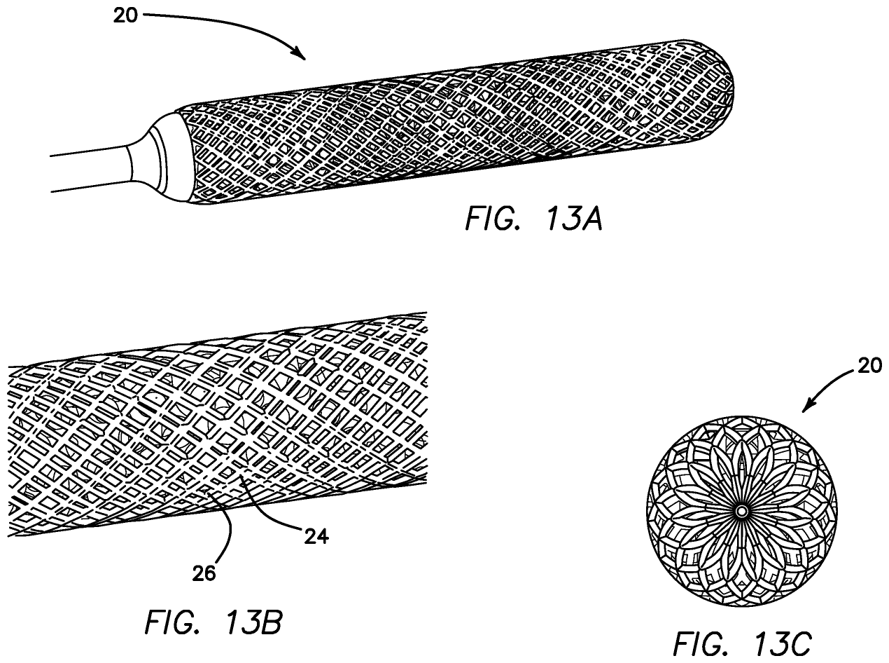

FIG. 13A-FIG. 13C illustrate a variety of different views of another variation of a sleeve or cover. In particular, the sleeve or cover illustrated in the figures comprise at least two different layers 24, 26. Each layer 24, 26 may be made of the same material or different materials. Each layer 24, 26 may have similar or different designs with respect to the access or pathways. There may be more than two layers. In any case, the combination of the layers 24, 26 can still provide an access or pathway between the outermost layer to the interior of the sleeve or cage so that specimen/tissue/fluids collected during use of the collection swab can be passed through to the interior to come into contact with a distal or tip portion (if available). In some embodiments, the layers 24, 26 can also provide another means of retaining the specimen/tissue/fluids within the sleeve or cage where the specimen/tissue/fluids may become trapped between two adjacent layers.

FIG. 14A-FIG. 14C illustrate another variation of the sleeve or cage. Whereas the earlier sleeve or cages described above had small access or passageways between the surface of the sleeve or cage, the sleeve or cage illustrated in the present figures are considerably larger. In particular, the sleeve or cage of FIG. 14A-FIG. 14C comprises at least two components which operate as overall support/braces 202 for the sleeve or cage aligned in parallel on each side of the sleeve or cage and the shaft of the collection swab. The support/brace 202 from a “plus”-like connection at the distal most end of the sleeve or cage. In addition to the support/brace 202, there are additional ribs 206 which are attached to each of the support/brace 202. These ribs 206 run perpendicular to the shaft of the collection swab. The combination of the support/brace 202 and the ribs 206 provide access or passageways 208 to the interior of the sleeve or cage.

In some embodiments, the sleeve or cage can be formed via molding as a single monolithic device along with the distal or tip portion (see FIG. 14B). In some embodiments, the sleeve or cage of FIG. 14A can be attached to the shaft of the collection swab via a connection between a proximal support ring (on the sleeve or cage) and a bolster (associated with the shaft of the collection swab).

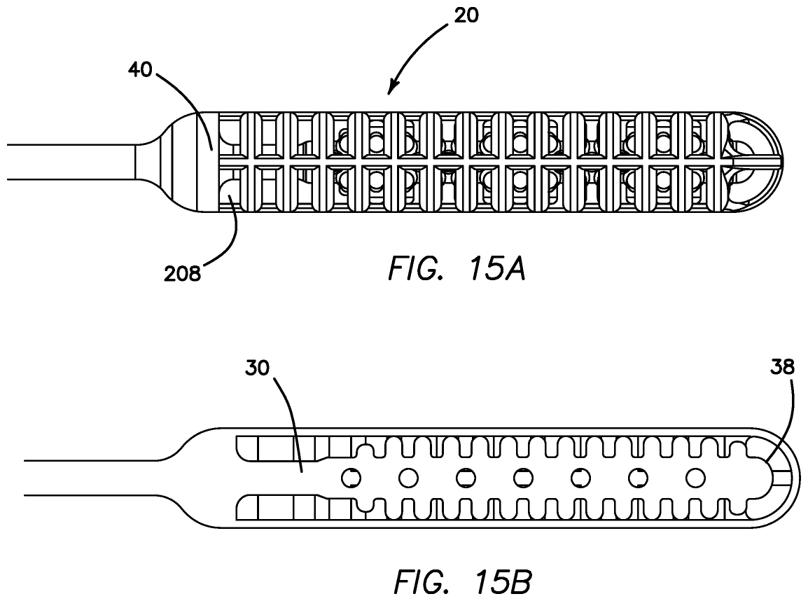

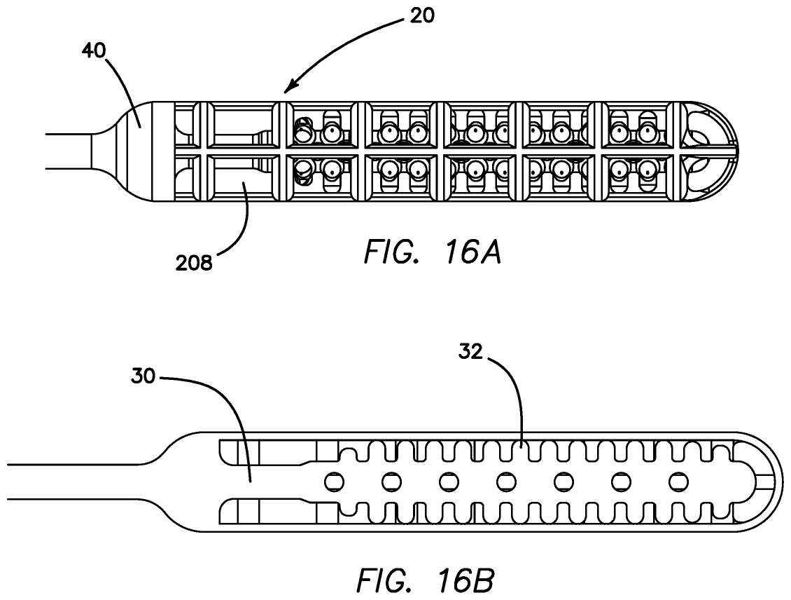

FIG. 15A and FIG. 15B illustrate another variation to the sleeve or cage. In particular, the sleeve or cage of these figures are similar to those illustrated in FIG. 14A-FIG. 14C except that the ribs of FIG. 15A and FIG. 15B are wider. In some cases, the ribs of FIG. 15A and FIG. 15B appear paddle-shaped. FIG. 16A and FIG. 16B illustrate another variation to the sleeve or cage. Here, the ribs are of a similar shape as those found in FIG. 15A and FIG. 15B. However, the difference between the sleeve or cage of FIG. 16A and FIG. 16B is that the spacing between consecutive ribs can be adjusted to be farther apart (thereby requiring less ribs over the entire length of the sleeve or cage). This creates even larger access or passageways for specimen/tissue/fluids to be collected and may provide easier access of such specimen/tissue/fluids to the enclosed distal or tip portion.

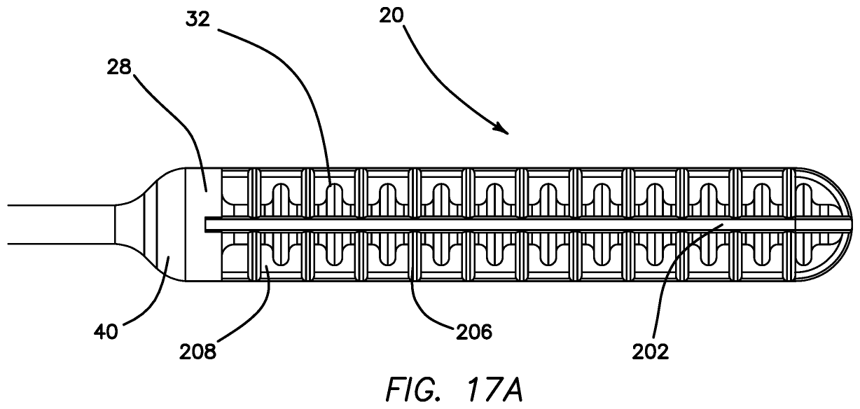

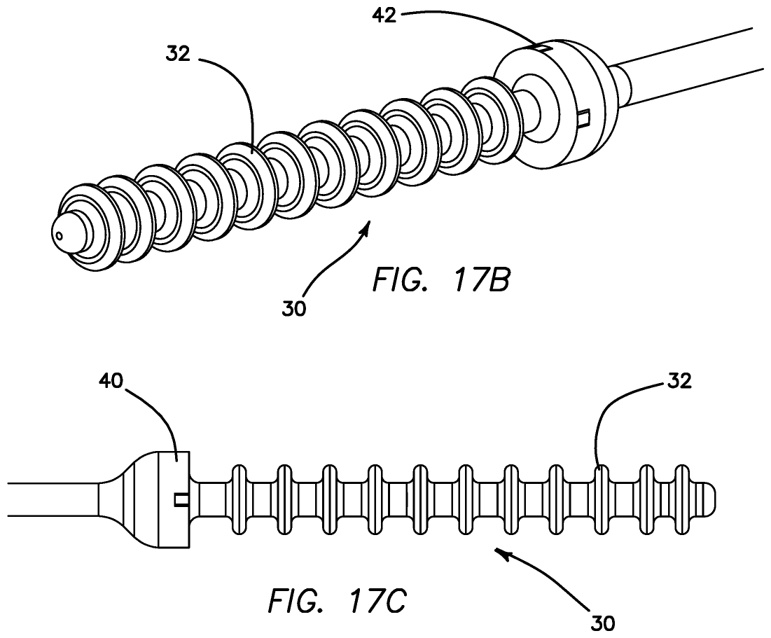

With respect to FIG. 17A-FIG. 17C, another variation of the sleeve or cage and enclosed distal or tip portion is illustrated. With respect to the sleeve or cage, the design is similar to the sleeve or cage illustrated in FIG. 14A-FIG. 14C. However, the enclosed distal or tip portion of the shaft has a variety of different features. First, the proximal end of the distal or tip portion includes a bolster 40 and associated indents 42 which are used to attach and connect the sleeve or cage 20 to the distal or tip portion of the shaft. The connection at the sleeve or cage 20 is provided with respect to the proximal support ring 28 which may have corresponding apertures which interface with the indents 42 of the bolster 40. This allows the sleeve or cage to be attached to the distal or tip portion and/or removed as needed.

In addition, the distal or tip portion of the shaft associated with the collection swab has a different design involving a number of protusions 32 which are circular. These protrusions 32 are spaced apart from each other along the length of the distal or tip portion. In this way the protrusions 32 can be used to scrape or dislodge specimen/tissue/fluids from a desired location as the collection swab is inserted and maneuvered at the desired location. The space between the protrusions 32 could be used to retain/absorb/capture the specimen/tissue/fluids when the collection swab is removed.

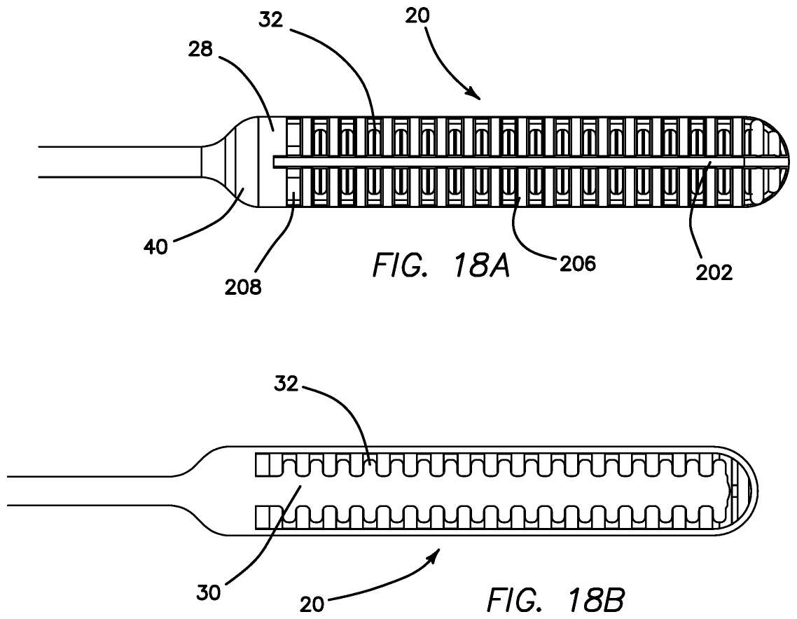

FIG. 18A and FIG. 18B illustrates another variation of the sleeve or cage. In this design, the arrangement of the support/braces 202 and the associated ribs 206 are similar to those seen in FIG. 14A-FIG. 14C. The main difference between the two variations is that the ribs 206 are spaced more closer to each other thereby requiring more ribs 206 along the length of the sleeve or cage. Furthermore, the access or passageways 208 are designed to be smaller compared to those found in the variation illustrated in FIG. 14A-FIG. 14C.

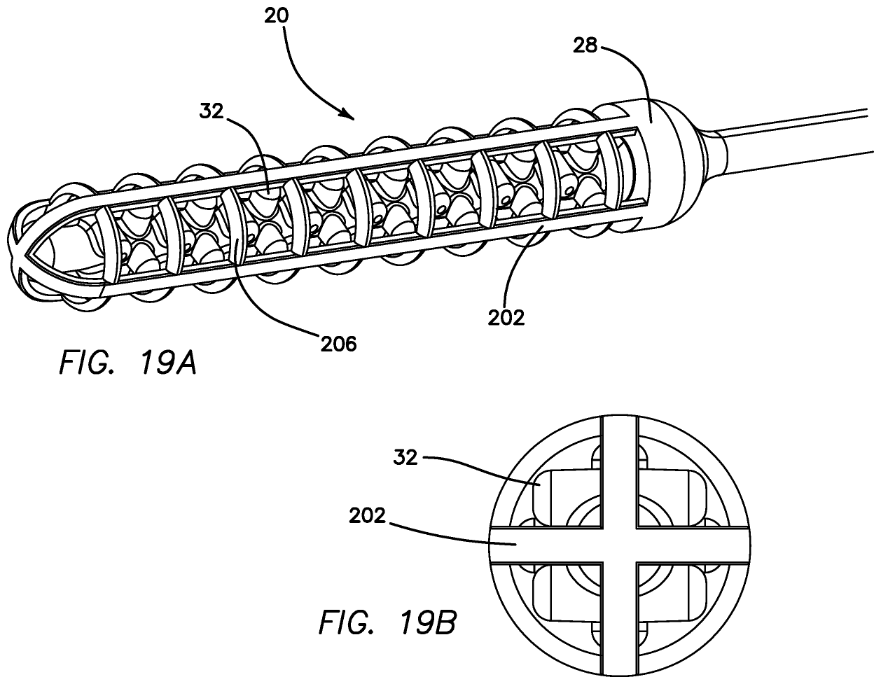

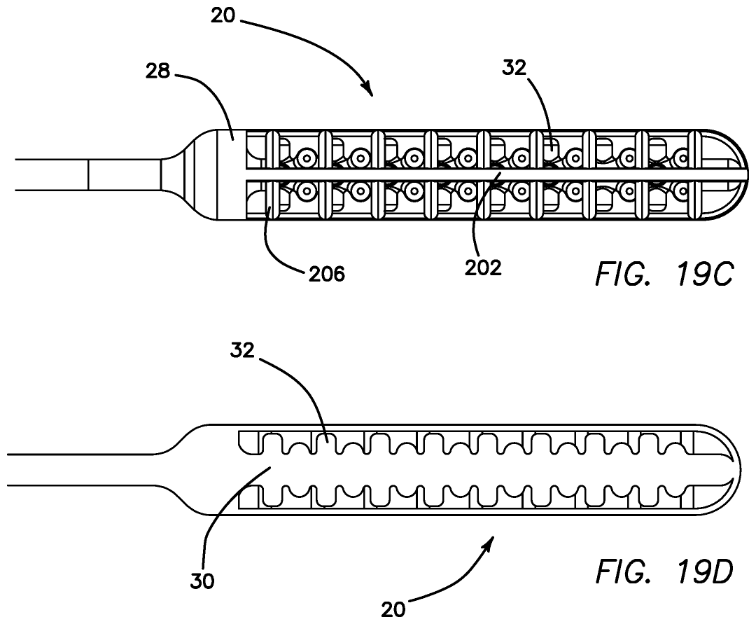

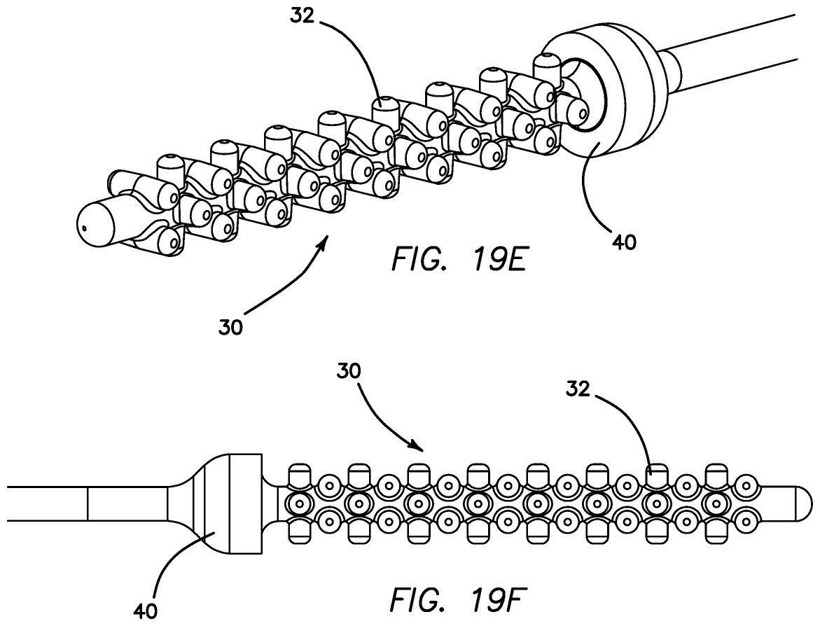

FIG. 19A-FIG. 19F illustrates another variation of the sleeve or cage and distal or tip portion. The sleeve or cage illustrated in FIG. 19A-FIG. 19D has support/braces 202 and ribs 206 arranged in a similar manner to those illustrated in FIG. 14-FIG. 14C. However, the design of the enclosed distal or tip portion can be seen as being different. In particular, on two opposite sides of the distal or tip portion is an alternating arrangement of protrusions 32 where some rows have two protrusions side by side while alternate rows only have a single protrusion 32. On the other two opposite sides, a single protrusion 32 is provided spaced evenly away from each other. The single protrusion 32 on the latter two sides are generally aligned with the row of protrusions only having a single protrusion (i.e. in between rows where two protrusions are next to each other). These protrusions 32 can have any number of apertures and/or through-holes used to collect and/or retain the specimen/tissue/fluid. Such collection and/or retention of the specimen/tissue/fluid may be similar to other variations discussed above. However, the difference in the sleeve or cage as well as the arrangement of the protrusions and/or apertures/through-holes can affect the rate and amount a particular collection swab can collect of a particular specimen/tissue/fluid as well as how much can be retained after the removal of the collection swab from the desired location.

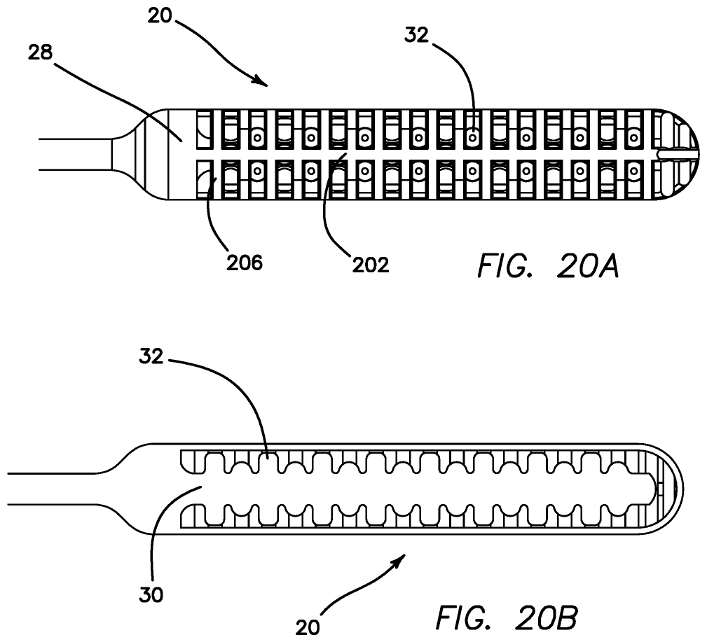

FIG. 20A and FIG. 20B illustrates another variation of the sleeve or cage. In particular, the sleeve or cage may have its support/braces 202 and ribs 206 formed as a single element. For example, the sleeve or cage can be manufactured via a molding process which provides the particular design as shown in the figures. This can be different from earlier embodiments where the support/braces 202 are produced separately and thus need to be attached with the ribs 206 when forming the sleeve or cage.

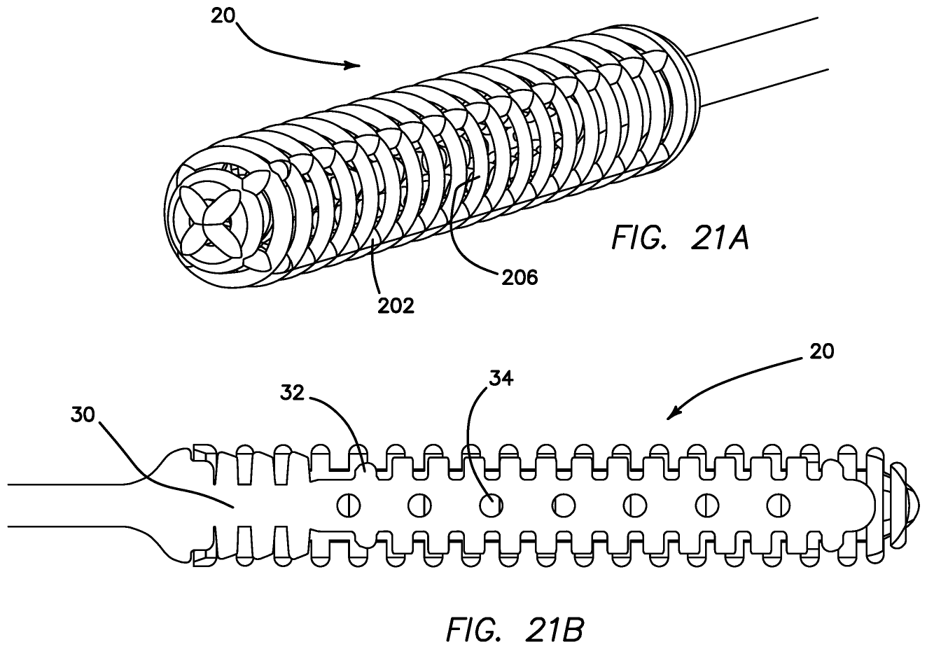

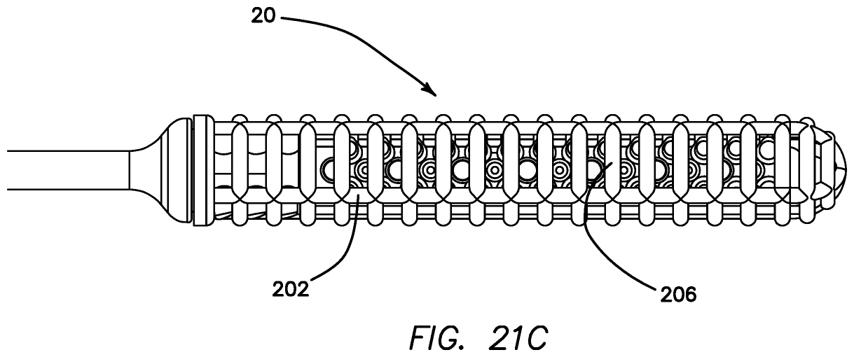

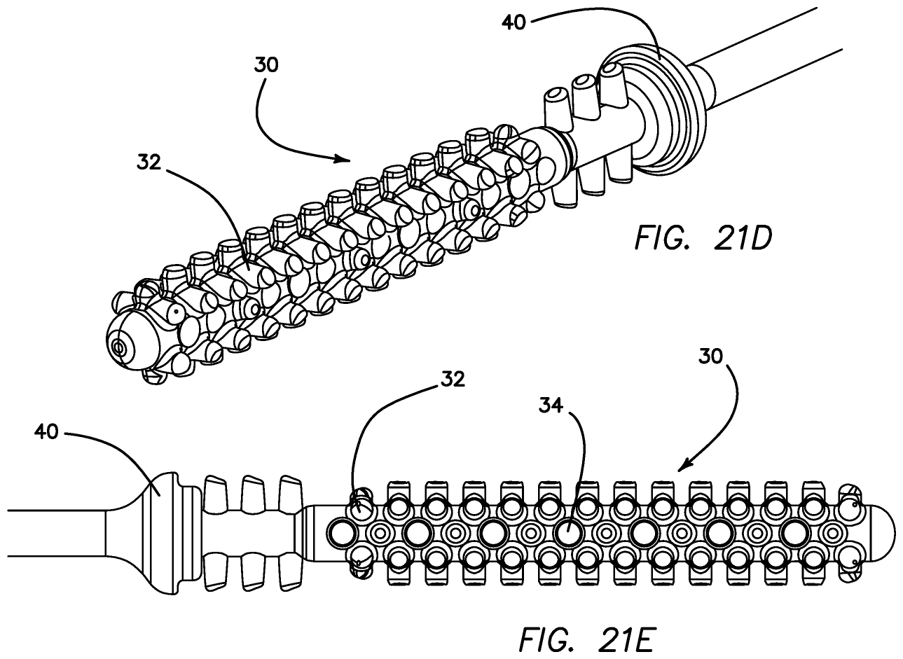

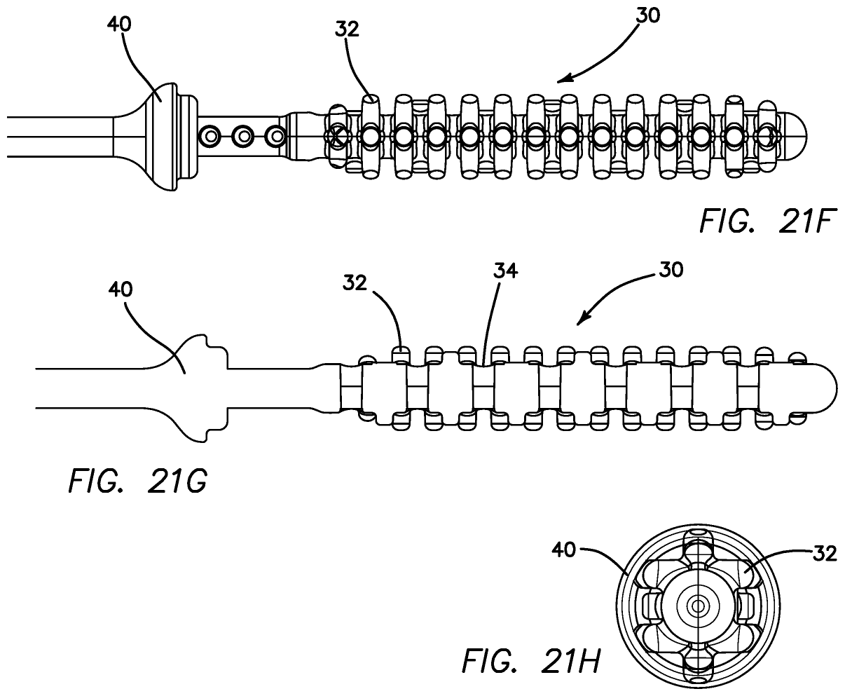

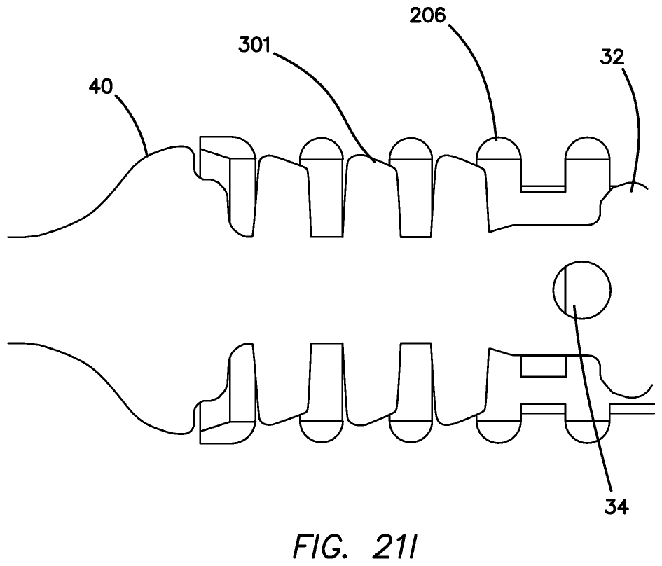

FIG. 21A-FIG. 21C illustrate another variation in the sleeve or cover. In particular, the sleeve or cover, though made of a combination of support/brace 202 and ring 206 elements similar to other embodiments described above, also have protrusions at various points of the sleeve or cover. In some embodiments, the protrusions found on the sleeve or cover can be positioned at the intersection of where the support/brace 202 and the ribs 206 are located. The protrusions can be additional elements that are added to the sleeve or cage. The protrusions can also be formed based on how the support/brace 202 and the ribs 206 are connected to each other when forming the sleeve or cage.

FIG. 21D-FIG. 21I illustrate another variation in the distal or tip portion. The distal or tip portion can be enclosed in a sleeve or cover, for example, as illustrated in FIG. 21A-FIG. 21 C though other sleeve or covers can also be compatible with the distal or tip portion in FIG. 21D FIG. 21I. The arrangement/design of the protrusions on the distal or tip portion may be similar to other variations discussed above (e.g., FIG. 19E-FIG. 19F). However, there are some differences compared to the earlier discussed designs which can affect the collection and retaining of the specimen/tissue/fluids. First, the protrusions may not all have uniform height. Furthermore, not all protrusions may have apertures. Lastly, towards the proximal end of the distal or tip portion of the shaft are three distinct proximal protrusions 301. These proximal protrusions 301 are spaced farther apart from each other compared to the other protrusions 32 found on the distal or tip portion. Furthermore, these proximal protrusions 301 can be designed to fit in between the access or passageways provided by the sleeve or cage. This allows at least a portion of the distal or tip portion to collect specimen/tissue/fluid directly from surfaces of the desired location when the proximal protrusions 301 come into contact with those surfaces as the collection swab is inserted and maneuvered.

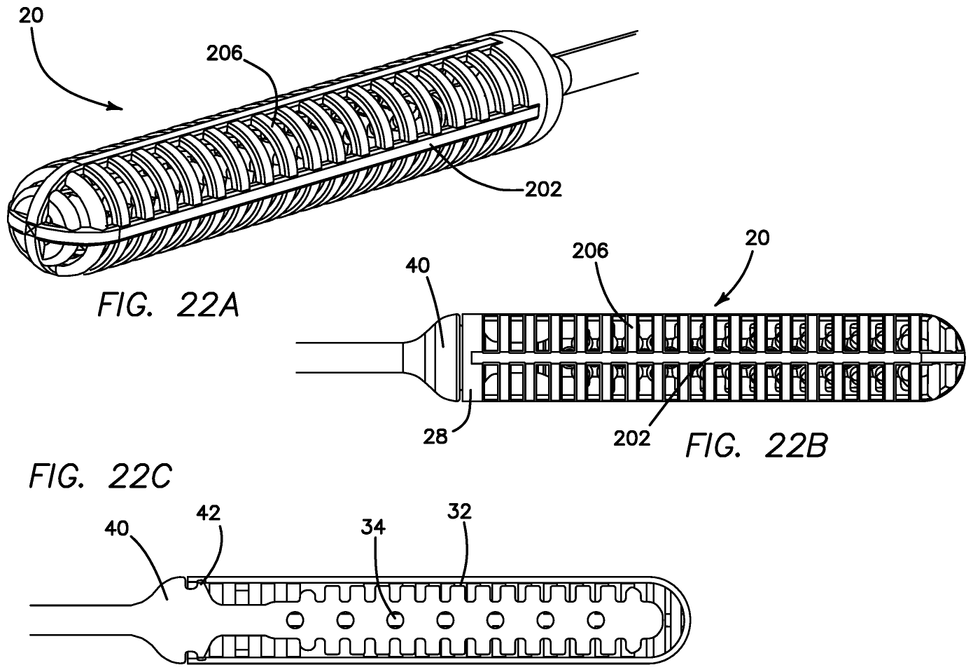

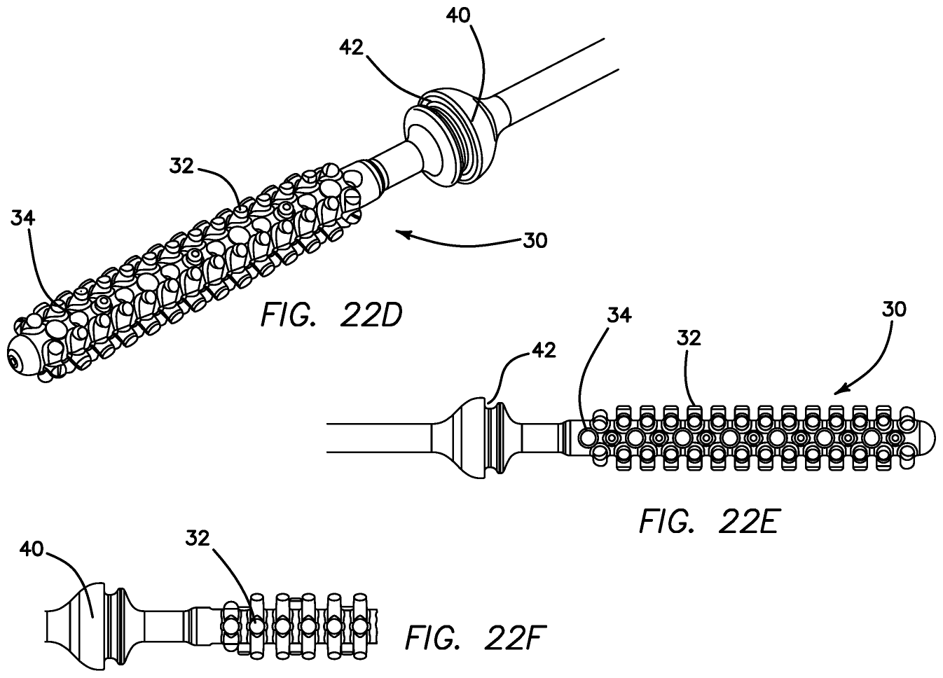

FIG. 22A-FIG. 22F illustrate another variation to the sleeve or cage and distal or tip portion of the present application. The sleeve or cage, as illustrated in FIG. 22A-FIG. 22C, has a similar design having a support/braces 202 at four points of the sleeve or cover and a plurality of ribs 206 along the length of the sleeve or cover.

What differs from the embodiments illustrated in the figures compared to the other embodiments discussed so far is with the distal or tip portion of the shaft associated with the collection swab. In particular, the distal or tip portion can be seen as having a distal portion associated with the protrusions while the proximal portion of the distal or tip portion is left bare (e.g., no protrusions are provided here). Rather, additional elements are added near the bolster 40 which are directed to allow for the attachment of a sleeve or cage to the distal or tip portion. For example, a channel or cutout 42 can be disposed around the bolster 40 which could provide a more secure fit between the sleeve or cage and the distal or tip portion. Furthermore, the connection between the sleeve or cage within the channel or cutout 42 could operate as a seal thereby providing a more secure way to retain specimen/tissue/fluid collected via the collection swab. The proximal support ring could have apertures or other features that would allow for the sleeve or cage to engage with the bolster 40 and/or channel or cutout 42 to assist in the connection and seal between the sleeve or cage and the distal or tip portion of the shaft.

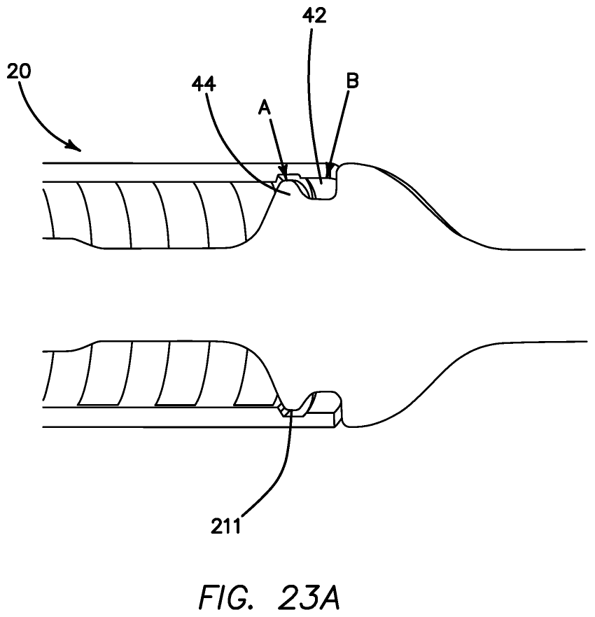

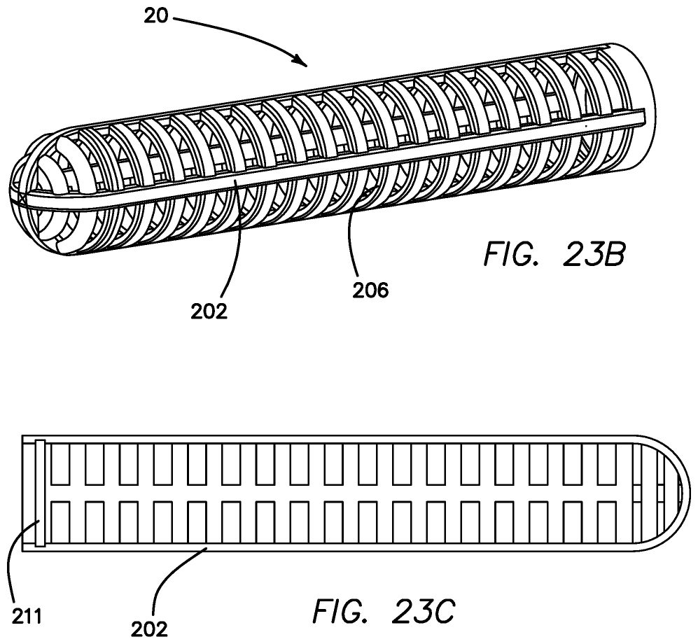

FIG. 23A-FIG. 23C illustrate an example sleeve or cage. The sleeve or cage has a channel 211 which comes in contact with the channel or cutout 42 of the distal or tip portion. Furthermore, there may be projections 44 that are associated with the bolster that can also connect with the channel 211 or other parts of the sleeve or cage. These connection points (labeled ‘A’ and ‘B’ in FIG. 23A) illustrate example connection points between the sleeve or cage and the distal or tip portion of the shaft.

As described above, the sleeve or cage can snap connect with the distal or tip portion of the shaft. Adhesives can also be used in order to secure the two elements together as well as provide a seal therebetween.

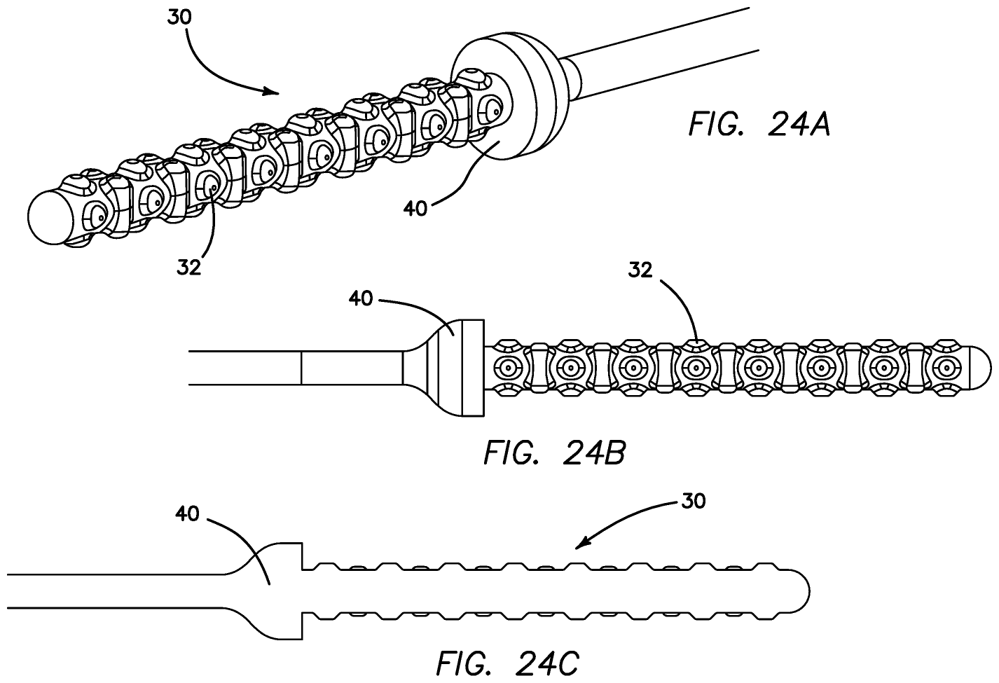

FIG. 24A-FIG. 24C illustrate another variation to the distal or tip portion of the shaft. The distal or tip portion of the shaft has two distinct arrangement of protrusions each having a through-hole. A first arrangement has a pair of protrusions facing in opposite directions. A second arrangement has four protrusions each facing in one of four different directions. The arrangement along the length of the distal or tip portion of the shaft alternates between these two arrangements.

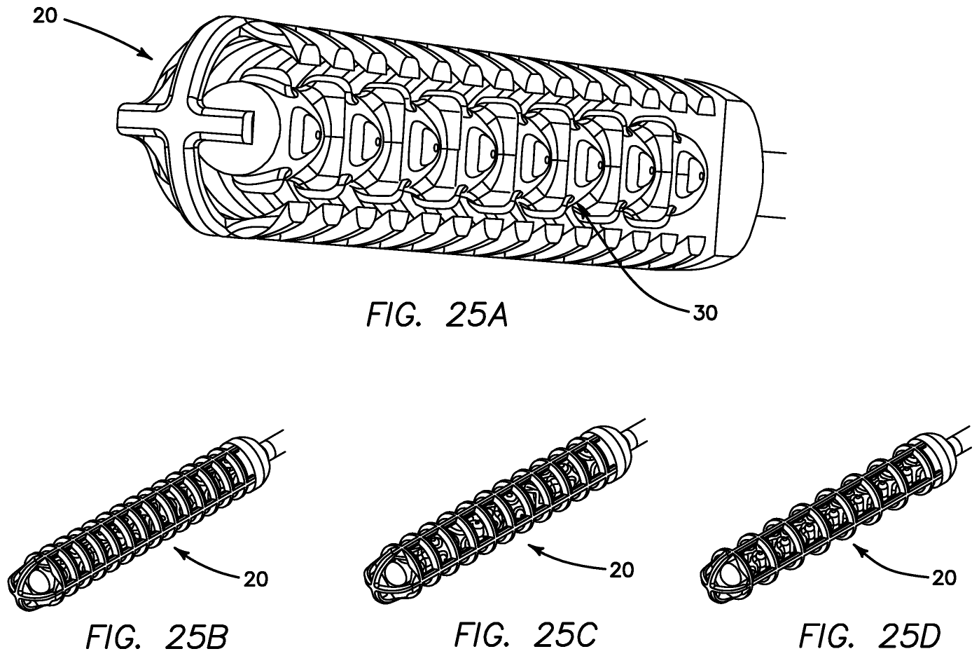

FIG. 25A-FIG. 25D illustrates another variation for the sleeve or cage. In particular, the width of the support/brace as well as the ribs can be modified to be as thick or as thin as needed. In these illustrations, the support/brace and ribs are made to be thicker in width compared to the other embodiments discussed within his application. This allows for less ribs to be used based on the size of the access or pathways implemented to allow the specimen/tissue/fluids to reach the distal or tip portion enclosed within.

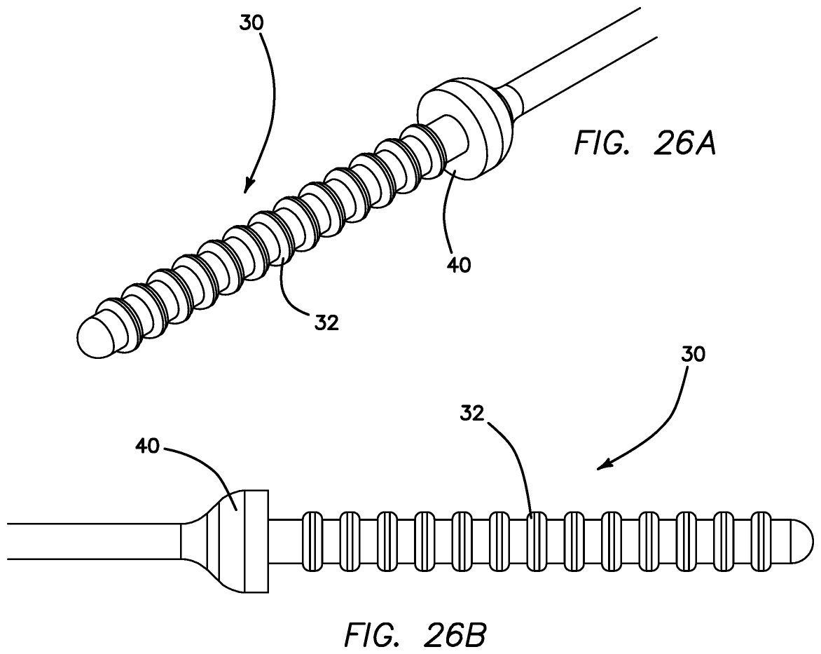

FIG. 26A and FIG. 26B illustrate another variation of the distal or tip portion. The distal or tip portion, as illustrated in the figure, has a number of circular protrusions along the length of the distal or tip portion. The protrusions compared to other embodiments (such as FIG. 17A-FIG. 17C) are not as high. As such, the overall collection swab may be capable of being fit into a smaller location. Furthermore, since the protrusions are not abruptly perpendicular to the shaft but rather smooth transitioning between the shaft and the protrusions, there may be less friction or resistance in tighter locations when inserting and maneuvering the collection swab.

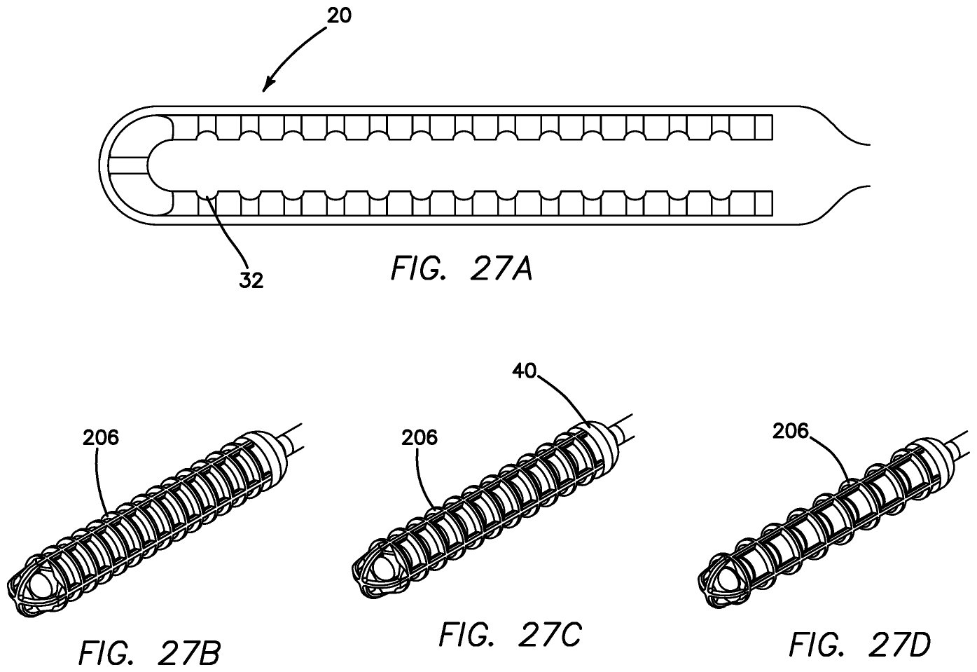

FIG. 27A-FIG. 27C illustrate variations in a sleeve or cage. In particular, the illustration shows how the number and spacing of the ribs along the length of the sleeve or cage can be varied. The access or passageway provided with the spacing between the ribs can control how much specimen/tissue/fluids can be captured as well as retained by the enclosed distal or tip portion.

FIG. 28A-FIG. 28B illustrate another variation to the sleeve or cage. The figure shows that the sleeve or cage can be manufactured (e.g., molded) to have a number of oval-shaped access or passageways. Furthermore the access or passageways can have a variety of different shapes

FIG. 29A-FIG. 29B illustrate another variation to the sleeve or cage. The sleeve or cage, again can be manufactured (e.g., molded) to have diamond-shaped access or passageways along the side of the sleeve or cage. The access or passageways can have different shapes towards the distal end of the sleeve or cage. For example, the sleeve or cage may include a distal ring 221 and a proximal ring 222. The proximal ring may help fortify the proximal end as well as provide a means to connect the sleeve or cage to the distal or tip portion of the shaft. The distal ring 221 would fortify the distal end of the sleeve or cage. The distal ring 221 may also have an access or passage present in the interior of the distal ring 221.

FIG. 30A and FIG. 30B illustrate another variation to the sleeve or cage. In particular, the sleeve or cage can have an arrangement of the support/brace and ribs such that the access or passageways therebetween are rectangular in shape.

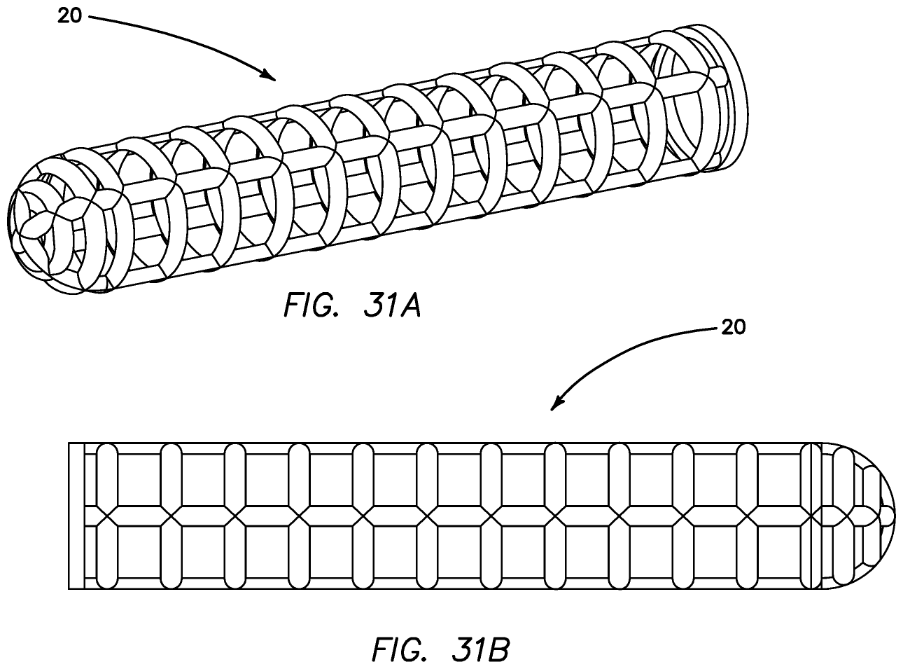

FIG. 31A and FIG. 31B illustrate another variation to the sleeve or cage. In this variation, the sleeve or cage can have an arrangement of the support/brace and ribs such that the access or passageways therebetween are more square-like. Furthermore the distal end of the sleeve or cage also has access or passageways.

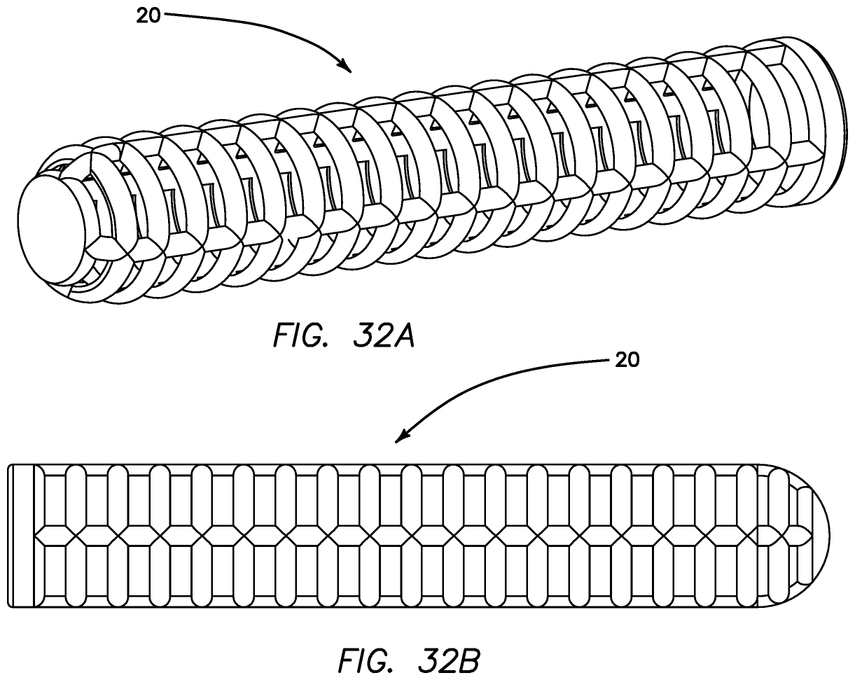

FIG. 32A and FIG. 32B illustrates another variation to the sleeve or cage. Similar to the sleeve or cage in FIG. 31A and FIG. 31B, a difference is present at the distal end of the sleeve or cage. Whereas FIG. 31A and FIG. 31B have access or passageways at the distal end, the sleeve or cage of FIG. 32A and FIG. 32B has a flat surface. The flat surface may be useful in pushing tissue aside to allow the user to maneuver the collection swab in the desired location.

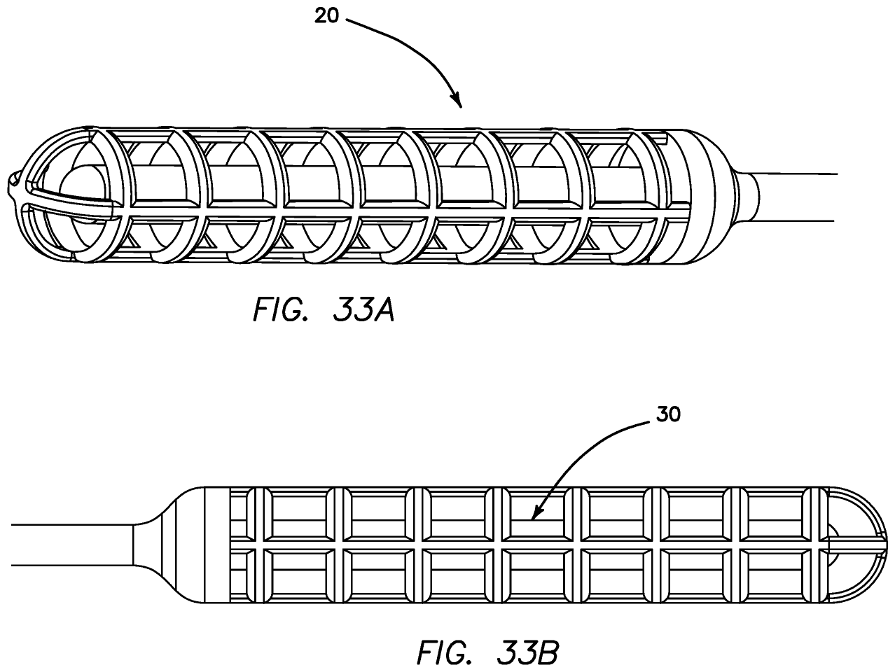

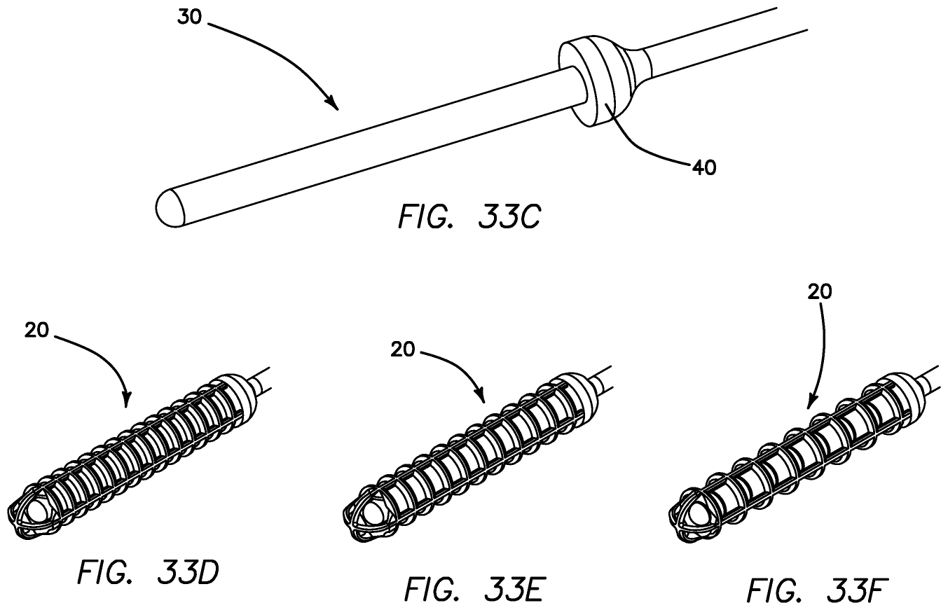

FIG. 33A-FIG. 33F illustrates another variation of the sleeve or cage and the distal or tip portion. In particular, the sleeve or cage may be any design discussed above. What is different, however, is the distal or tip portion. In particular, the distal or tip portion is smooth (i.e. has no protrusions or apertures) as seen in FIG. 33C. In this embodiment, the distal or tip portion may be made of a variety of different materials that provide differing absorption and retention of specimen/tissue/fluids dislodged and collected by the sleeve or cage thereby not needing the use of any protrusions or apertures. Different materials can be used based on the type of specimen/tissue/fluids being captured and retained. Furthermore, the materials can also affect the amount that can be collected and retained (e.g., some materials may be more absorbent but provide difficulty in removing from the collection swab after).

FIG. 34A-FIG. 35C and FIG. 36B-FIG. 36E illustrate similar variations for the collection swab. In particular, the collection swab comprises a shaft which is connected to a sleeve or cage. The sleeve or cage can have any number of different designs as illustrated and/or discussed above in this application. However, what is different in these embodiments is that the shaft does not have a distal or tip portion. In other words, the interior space of the sleeve or cage which has been described as enclosing a distal or tip portion is instead empty.

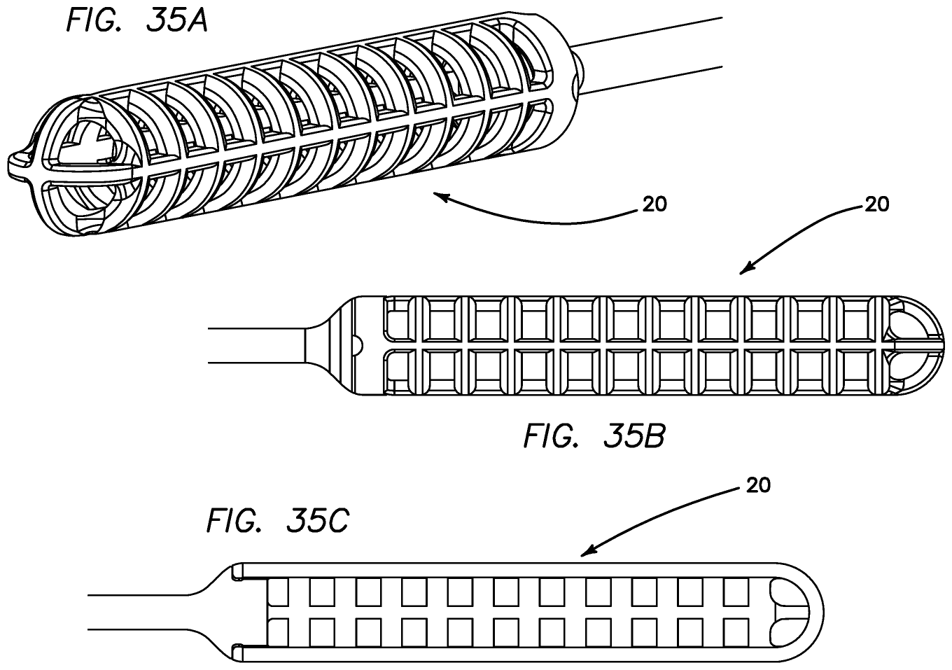

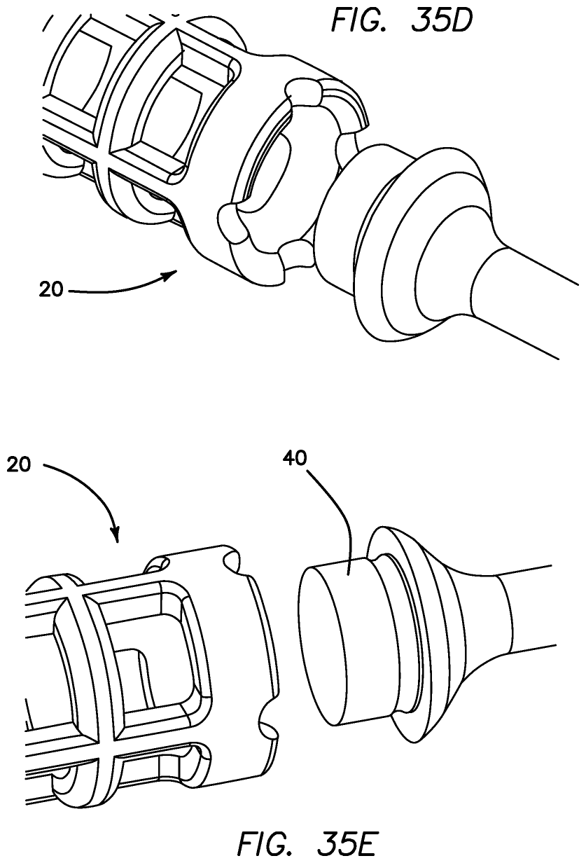

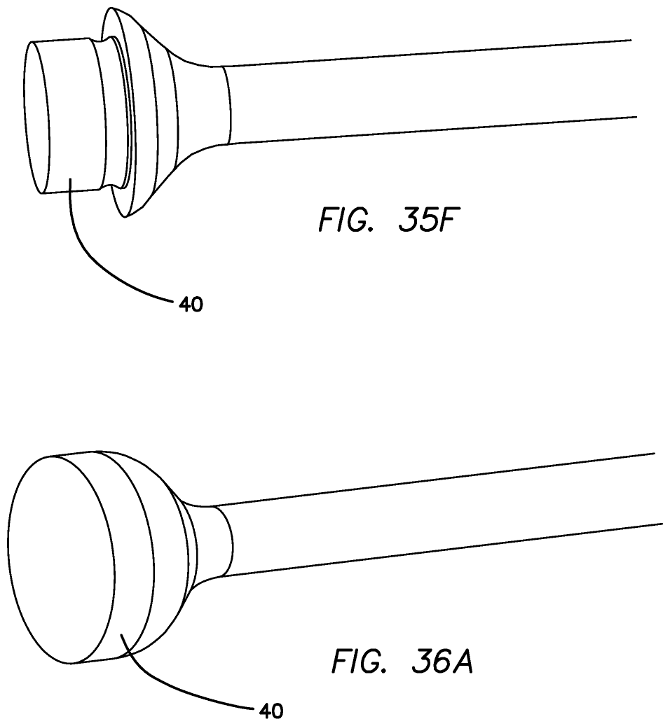

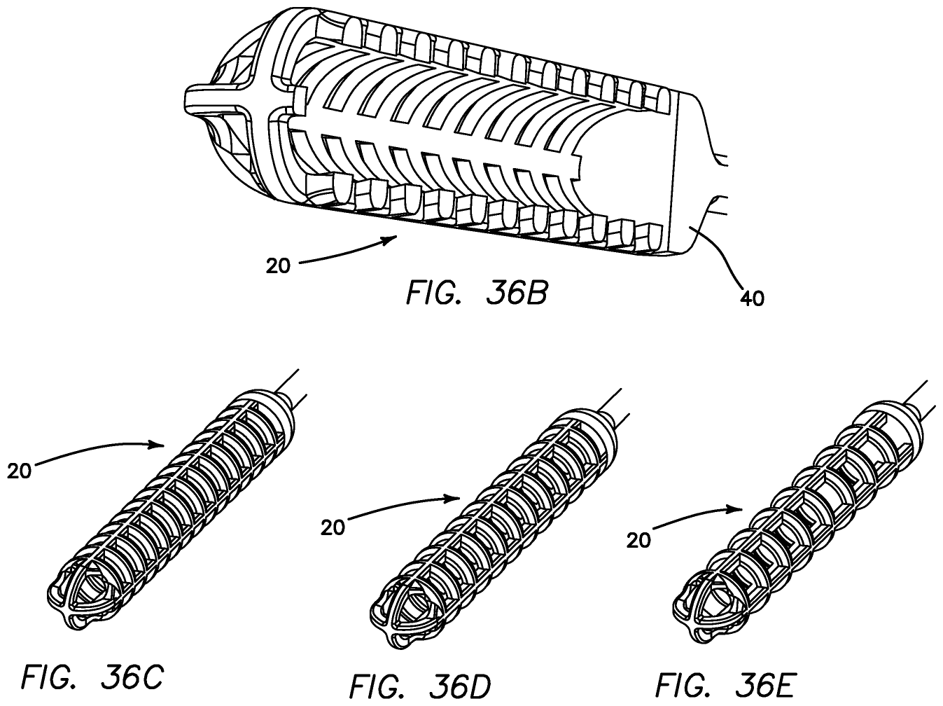

FIG. 35D-FIG. 36A illustrate how the sleeve or cage can be connected to the shaft of the collection swab. As discussed above, there may be a variety of options that can be used to connect the sleeve or cage to the shaft. These figures illustrate the connection of the bolster (associated with the shaft) and the proximal support ring of the sleeve or cage. Furthermore, the proximal support ring may have any number of features (e.g., protrusions, indents) that can be used to interface with features associated with the bolster (e.g., channel, cuts). The sleeve or cage may be snapped into and held together based on the features of the sleeve or cage and shaft. However, there may be variations where adhesives can also be used to secure the sleeve or cage to the shaft.

These variations of the collection swab may be used if the sleeve or cage is designed to collect and retain the specimen/tissue/fluid, there may not be a need for a distal or tip portion. Thus, by removing the unnecessary portion of the collection swab, this can simplify the manufacture of the collection swab.

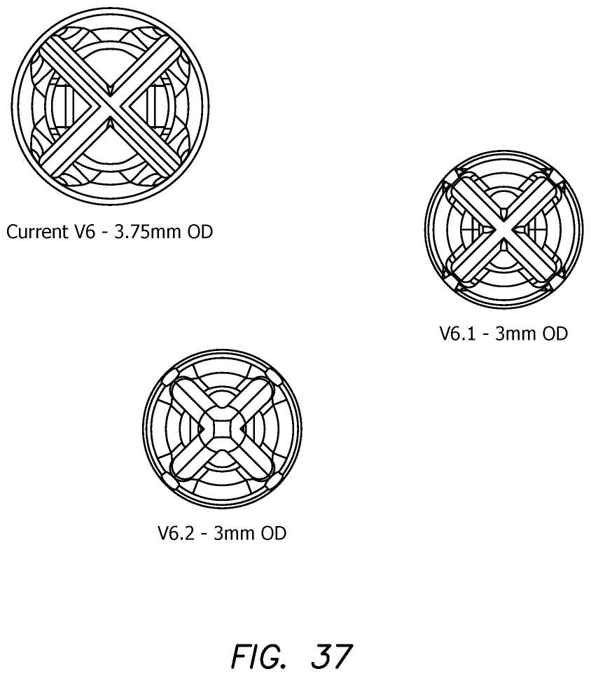

FIG. 37 illustrate variations on the design of the distal point of the sleeve or cage. The design chosen for a particular collection swab, as illustrated in the figure, can vary based on the arrangement and size/shape of the support/brace and accompanying ribs associated with the sleeve or cage. Some variations may have larger access or passageways on the distal point of the sleeve or cage. The design chosen can also be made to accommodate the enclosed distal or tip portion (and associated protrusions and/or apertures). For example, the ribs and/or support/brace may be aligned with the location of the protrusions and/or apertures associated with the distal or tip portion to facilitate in the capture and retention of specimen/tissue/fluids collected as the collection swab is inserted into the desired location.

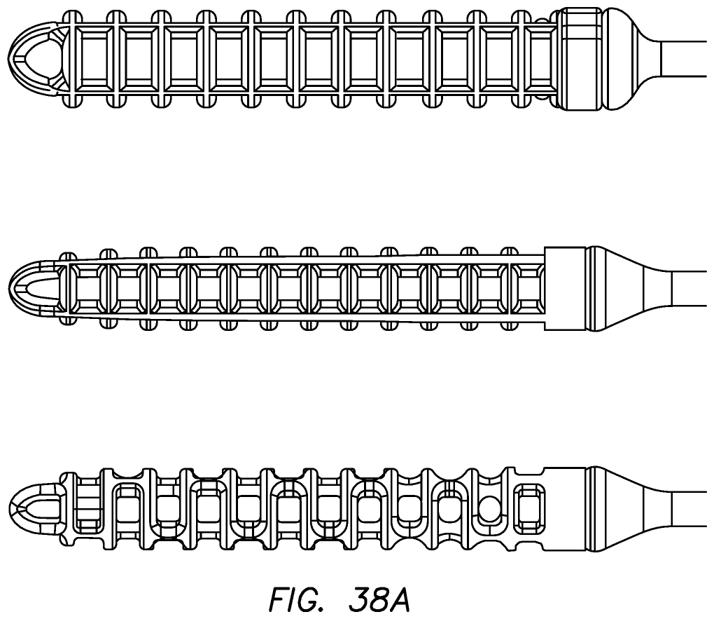

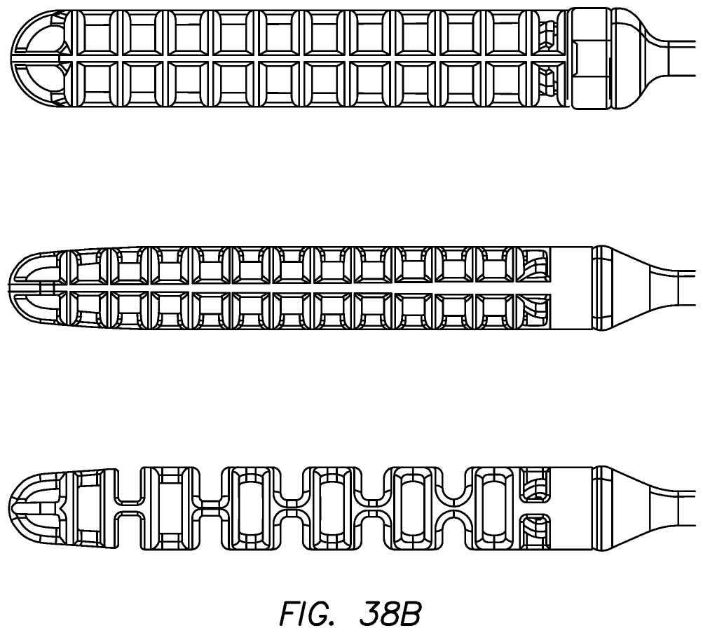

FIG. 38A-FIG. 40C illustrate additional variations associated with the sleeve or cage. Different shapes and sizes for the overall sleeve or cage as well as the support/brace and ribs provide different benefits. For example, some sleeve or cages may be more tapered or cone-shaped in order to facilitate entry of the sleeve or cage into the desired location that may be narrow (e.g., nasal passage).

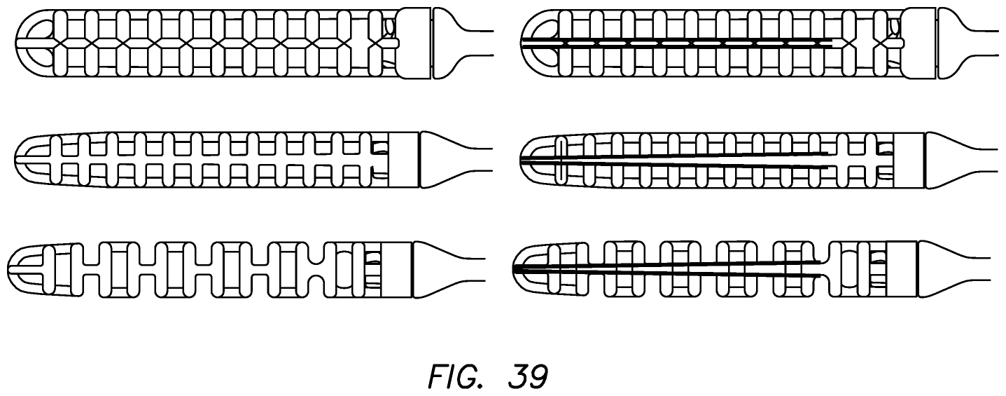

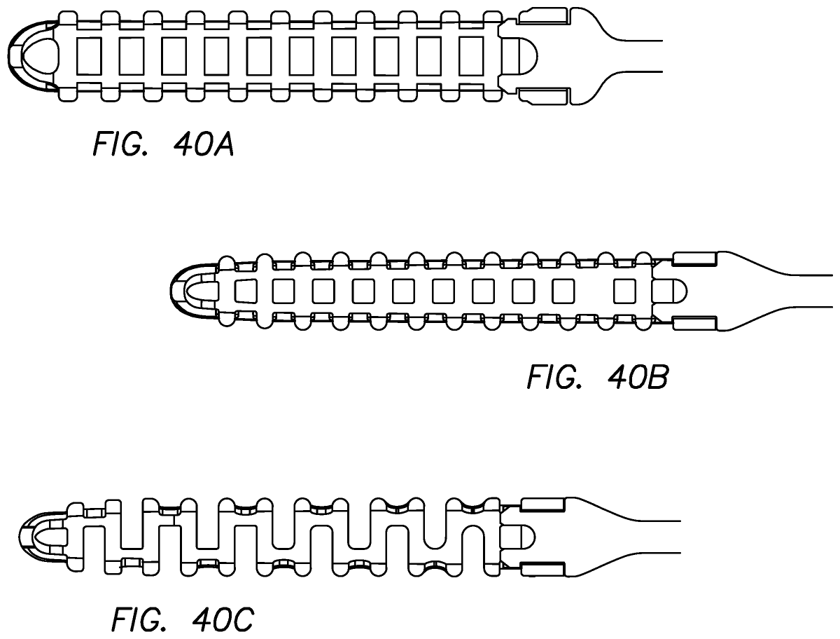

Different size ribs are also used based on the location along the sleeve or cage. Smaller and thinner ribs can be used at the distal end of the sleeve or cage in order to provide the ability of the sleeve or cage to flex and bend as the collection swab is inserted and navigated within the desired location. Meanwhile thicker ribs can be used at the proximal end of the sleeve or cage in order to provide a more sturdy foundation as the sleeve or cage flexes or bends. Other methods of allowing for flexibility/sturdiness for the sleeve or cage can also be realized based on the reduction in the number of support/braces implemented (see the third variations in FIG. 38B and FIG. 39, FIG. 40C).

The above description is provided to enable any person skilled in the art to make and use the devices or systems and perform the methods described herein and sets forth the best modes contemplated by the inventors of carrying out their inventions. Various modifications, however, will remain apparent to those skilled in the art. It is contemplated that these modifications are within the scope of the present disclosure. Different embodiments or aspects of such embodiments may be shown in various figures and described throughout the specification. However, it should be noted that although shown or described separately each embodiment and aspects thereof may be combined with one or more of the other embodiments and aspects thereof unless expressly stated otherwise. It is merely for easing readability of the specification that each combination is not expressly set forth.

Although the present invention has been described in certain specific aspects, many additional modifications and variations would be apparent to those skilled in the art. It is therefore to be understood that the present invention may be practiced otherwise than specifically described, including various changes in the size, shape and materials, without departing from the scope and spirit of the present invention. Thus, embodiments of the present invention should be considered in all respects as illustrative and not restrictive.

What is claimed:

1. A collection swab comprising:
a handle portion located at a proximal end of the collection swab, the handle portion configured for a user to maneuver the collection swab within a desired location; and
a distal or tip portion connected to the handle portion and configured to collect a specimen from the desired location, wherein the distal or tip portion is configured to capture the specimen from the desired location as the collection swab is maneuvered within the desired location and retain the specimen when the collection swab is removed from the desired location.

2. The collection swab of claim 1, wherein the distal or tip portion comprises a plurality of protrusions.

3. The collection swab of claim 2, wherein the plurality of protrusions are circular in shape around the longitudinal axis of the collection swab, and wherein the distal or tip portion further comprises grooves defined by two consecutive circular protrusions that are configured to retain the specimen captured by each circular protrusion.

4. The collection swab of claim 2, wherein each of the plurality of protrusions are dome shaped.

5. The collection swab of claim 4, wherein one or more of the dome-shaped protrusions have through-holes at a tip.

6. The collection swab of claim 4, wherein one or more of the dome-shaped protrusions have an aperture attached at a tip.

7. The collection swab of claim 2, wherein the distal or tip portion further comprise a plurality of apertures arranged in between each of the plurality of protrusions.

8. The collection swab of claim 7, wherein a height of the plurality of apertures differ from a height of the plurality of protrusions.

9. The collection swab of claim 2, wherein the plurality of protrusions comprise two subset of protrusions, and wherein a height associated with a first subset of protrusions is smaller than a height associated with a second subset of protrusions.

10. The collection swab of claim 1 further comprising a sleeve or cage that encompasses the distal or tip portion of the collection swab.

11. The collection swab of claim 10, wherein the sleeve or cage further comprises a proximal support ring, and wherein the sleeve or cage is removably connected to the distal or tip portion via the proximal support ring.

12. The collection swab of claim 10, wherein the sleeve or cage is mechanically snapped together with the distal or tip portion of the collection swab.

13. The collection swab of claim 10, wherein the sleeve or cage is glued together with the distal or tip portion of the collection swab.

14. The collection swab of claim 10, wherein the sleeve or cage is configured to flex as the collection swab is maneuvered in the desired location, a degree of flex being provided for the sleeve or cage based on the sleeve or cage having a tapered or cone-shaped design.

15. The collection swab of claim 10, wherein the sleeve or cage is configured to flex as the collection swab is maneuvered in the desired location, a degree of flex being provided for the sleeve or cage based on the sleeve or cage using thin ribs.

16. The collection swab of claim 10, wherein the sleeve or cage further comprises a distal ring configured to fortify the distal end of the sleeve or cage.

17. The collection swab of claim 1 wherein the sleeve or cage comprises a plurality of layers, the plurality of layers defining a plurality of access or passageway to the interior space of the sleeve or cage, and wherein the plurality of layers are also configured to retain at least a portion of the specimen between two consecutive layers.

18. The collection swab of claim 1, further comprising two or more sleeves or cages, wherein each of the two or more sleeves or cages enclose the distal or tip portion of the collection swab.

19. A collection swab comprising:
a handle portion located at a proximal end of the collection swab, the handle portion configured for a user to maneuver the collection swab within a desired location;
a distal or tip portion connected to the handle portion and configured to collect and retain a specimen from the desired location; and
a sleeve or cage that encloses the distal or tip portion in an interior space and is connected to the distal or tip portion, wherein the sleeve or cage comprise:
a plurality of ribs that define the interior space of the sleeve or cage, wherein the plurality of ribs are configured to capture and retain the specimen from the desired location as the collection swab is maneuvered within the desired location, and
a plurality of access or passageways defined by space between consecutive ribs, each access or passageway providing a path for the specimen captured by the plurality of ribs to travel to the interior space of the sleeve or cage, wherein the specimen within the interior space of the sleeve or cage are captured and retained by the distal or tip portion enclosed therein.

20. A collection swab comprising:
a handle portion located at a proximal end of the collection swab, the handle portion configured for a user to maneuver the collection swab within a desired location; and
a sleeve or cage that is connected to the handle portion, wherein the sleeve or cage comprise:
a plurality of ribs that define the interior space of the sleeve or cage, wherein the plurality of ribs are configured to capture and retain the specimen from the desired location as the collection swab is maneuvered within the desired location, and
a plurality of access or passageways defined by space between consecutive ribs, each access or passageway providing a path for the specimen captured by the plurality of ribs to travel to the interior space where the specimen can be retained.
